(19) United States
(12) Patent Application Publication
(10) Pub. No.: US 2022/0411360 A1
(43) Pub. Date: Dec. 29, 2022

(54) METABOLITE-BASED POLYMERS AND MICROPARTICLES FOR DELIVERY OF THERAPEUTIC AGENTS AND TISSUE REGENERATION

(71) Applicant: Arizona Board of Regents on behalf of Arizona State University, Scottsdale, AZ (US)

(72) Inventors: Abhinav Acharya, Tempe, AZ (US); Kaushal Rege, Chandler, AZ (US)

(21) Appl. No.: 17/775,372

(22) PCT Filed: Nov. 9, 2020
(86) PCT No.: PCT/US20/59610

§ 371 Date: May 9, 2022

Related U.S. Application Data
(60) Provisional application No. 62/933,391, filed on Nov. 9, 2019.

(51) Int. Cl. C07C 53/126; A61K 47/69

(52) U.S. Cl. CPC C07C 53/126; A61K 47/6927

(57) ABSTRACT

The present invention provides polymers, particles, and compositions thereof that selectively and efficiently deliver various therapeutic agents, such as metabolites, to a cell. The present invention further relates to methods relating to the said polymers, particles, and compositions for enhancing biological tissue growth (e.g. biological tissue regeneration in wound healing) in a subject. The present invention additionally provides kits that find use in the practice of the methods of the invention.

### CROSS-REFERENCE TO RELATED APPLICATIONS

This application claims priority to and the benefit of U.S. Provisional Application No. 62/933,391, filed Nov. 9, 2019, the disclosure of which is incorporated herein by reference in its entirety.

### BACKGROUND OF THE INVENTION

There is a rich history of successful drug delivery carriers made of biodegradable biomaterials. Examples of such carriers include polyesters (e.g., poly (lactic-co-glycolic) acid (PLGA), which is used in applications ranging from cancer to autoimmunity) and bi-lipid layer carriers (e.g., liposomes). Notably, these biomaterials may degrade into metabolic by-products, which are capable of modulating the function of immune cells. For example, the degradation product of the drug delivery carrier poly(lactic acid) is lactic acid (a by-product of glycolysis), which may be able to directly suppress immune cells, such as dendritic cells (DCs; specialized immune cells responsible for inducing adaptive immune responses), macrophages (phagocytes responsible for removing debris), and T-cell lymphocytes (responsible for mounting immune responses against foreign materials). Interestingly, there are several metabolites that are known to modulate function of immune cells, including succinate, which activates DCs and leads to adaptive immune response; citrate, which induces pro-inflammatory cytokines and reactive oxygen species; and alpha-ketoglutarate (αKG or aKG), which induces alternate activation (immunosuppressive phenotype) in macrophages through metabolic reprogramming. However, delivery of αKG to modulate the metabolism of immune cells is non-trivial, as this molecule gets metabolized quickly, diffuses away from the injection site, and therefore needs to be provided via multiple injection.

Thus, there is a need in the art for improved methods of delivery of therapeutic agents (e.g., αKG) for tissue regeneration and/or treatment of diseases or disorders. The present invention satisfies this unmet need.

### BRIEF SUMMARY OF THE INVENTION

The present invention relates, in part, to a compound or salt thereof comprising the structure of Formula (I)

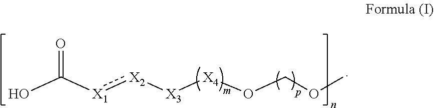

In some embodiments, each occurrence of X₁ and X₂ is independently C═R₁, CR₂, or CR₃R₄. In some embodiments, each occurrence of X₃ and X₄ is independently C═R₁ or CR₃R₄.

In some embodiments, the bond between X₁ and X₂ is a single bond or a double bond. In some embodiments, when the bond between X₁ and X₂ is a single bond, X₁ and X₂ are each independently C═R₁ or CR₃R₄. In one embodiment, when the bond between X₁ and X₂ is a double bond, X₁ and X₂ are each CR₂.

In some embodiments, R₁ is O, NH, or S. In one embodiment, R₁ is O. In some embodiments, each occurrence of R₂, R₃, and R₄ is independently hydrogen, hydroxyl, carboxyl, alkyl, cycloalkyl, heterocycloalkyl, aryl, or heteroaryl. In some embodiments, R₂ is hydrogen or hydroxyl. In some embodiments, R₃ is hydrogen, hydroxyl, or carboxyl. In some embodiments, R₄ is hydrogen, hydroxyl, or carboxyl.

In some embodiments, m is an integer represented by 0, 1, 2, or 5. In some embodiments, n is an integer from 1 to 1000. In some embodiments, p is an integer from 1 to 50.

In some embodiments, p is an integer from 2-16.

In some embodiments, the compound comprising the structure of Formula (I) is a compound comprising the structure of:

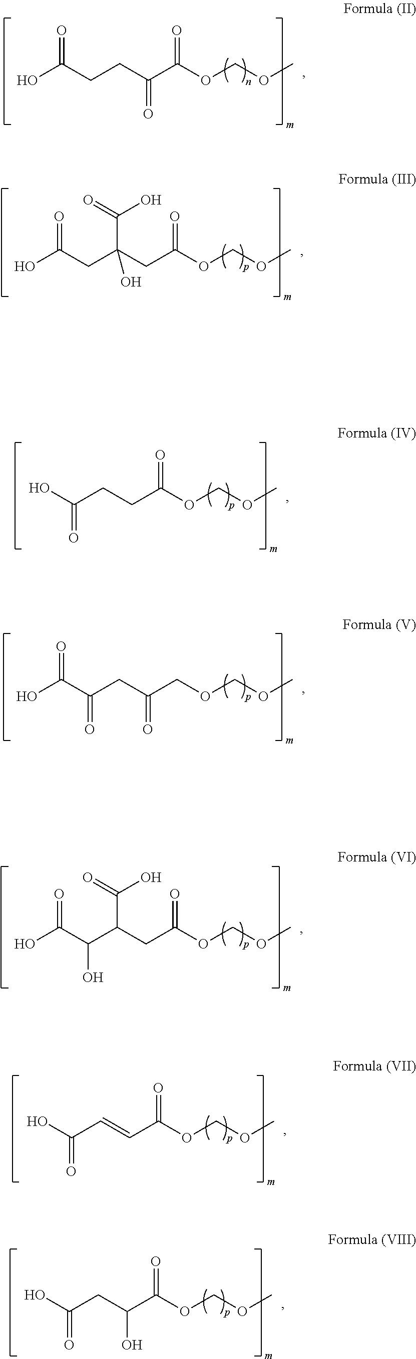

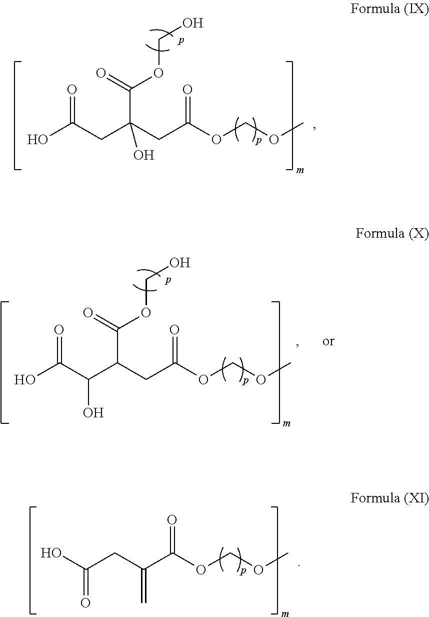

In some embodiments, each occurrence of p is an integer from 1 to 15. In some embodiments, each occurrence of m is an integer from 1 to 1000.

In one embodiment, the compound comprising the structure of Formula (I) is a compound comprising the structure of Formula (II)

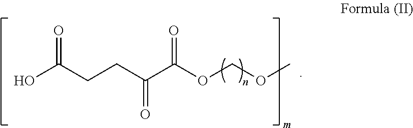

In one embodiment, n is an integer represented by 10.

In one aspect, the present invention also relates, in part, to a method of making a polymer comprising the structure of Formula (I):

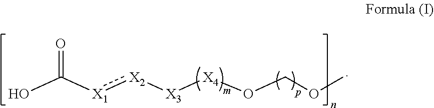

In one embodiment, the method comprises reacting a compound or salt thereof comprising the structure of Formula (Ia) and a compound or salt thereof comprising the structure of Formula (Ib)

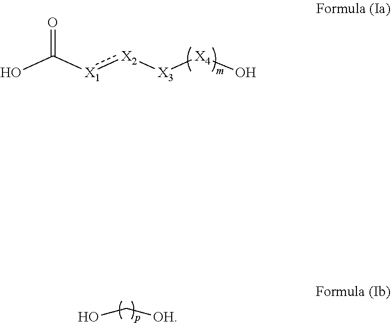

In another aspect, the present invention relates, in part, to a method of forming particles. In various embodiments, the method comprises the steps of: (a) mixing the compound comprising the structure of Formula (I) with an oil solvent and water solvent; and (b) forming the particle in the water-oil emulsion.

In another aspect, the present invention relates, in part, to a particle comprising at least one compound comprising the structure of Formula (I). In some embodiments, the particle has an average size of about 0.01 μm to about 1000 μm.

In one embodiment, the particle encapsulates at least one therapeutic agent. In one embodiment, the compound comprising the structure of Formula (I) encapsulates the at least one therapeutic agent.

In another aspect, the present invention relates, in part, to a method of delivering a therapeutic agent to a cell in a subject in need thereof.

In yet another aspect, the present invention relates, in part, to a method of enhancing biological tissue growth in a subject in need thereof.

In another aspect, the present invention relates, in part, to a method of enhancing biological wound healing or wound closure in a subject in need thereof.

In various embodiments, the method comprises administering at least one particle of the present invention to the subject. In one embodiment, the particle encapsulates the therapeutic agent. In some embodiments, the particle releases a therapeutic agent inside or outside the cell.

In one embodiment, the therapeutic agent is an anti-inflammatory therapeutic agent. In one embodiment, therapeutic agent is a metabolite. In some embodiments, the metabolite is α-ketoglutarate (αKG), succinic acid, citric acid, spermidine, itaconic acid, or any combination thereof.

In one embodiment, the particle is administered orally, topically, intravenously, intraperitoneally, or intramuscularly to the subject.

### BRIEF DESCRIPTION OF THE DRAWINGS

The following detailed description of various embodiments of the invention will be better understood when read in conjunction with the appended drawings. For the purpose of illustrating the invention, there are shown in the drawings illustrative embodiments. It should be understood, however, that the invention is not limited to the precise arrangements and instrumentalities of the embodiments shown in the drawings.

FIG. 1 depicts a schematic representation of how central-carbon metabolite based polymers modulate the function of dendritic cells via modulating intracellular metabolites of the cells.

FIG. 2, comprising FIG. 2A through FIG. 2E, depicts the synthesis and characterization of polymeric particles of non-activating metabolites are synthesized. FIG. 2A depicts a schematic representation of the condensation reaction that was used to generate poly-alpha-ketoglutarate (pαKG or paKG) polymers. FIG. 2B depicts representative ¹H NMR spectra that confirmed the presence of 1,10-decanediol and αKG in pαKG and showed the release of αKG from particles at day 10. FIG. 2C depicts electron microscopy micrograph of microparticles that were generated from pαKG. FIG. 2D depicts the average particle size of microparticles that were generated from pαKG, as determined by dynamic light scattering. FIG. 2E depicts that the pαKG particles degrade over a period of time via hydrolysis of the ester bond as determined by weight loss experiments over 60 days.

FIG. 3, comprising FIG. 3A through FIG. 3E, depicts representative data demonstrating that central-carbon metabolite-based microparticles release metabolites in a sustained manner. FIG. 3A depicts a schematic representation of the pαKG synthesis. FIG. 3B depicts a scanning electron microscope micrograph of pαKG microparticles. FIG. 3C depicts dynamic light scattering size distribution results of pαKG microparticles (average=4138±142). FIG. 3D depicts representative release kinetic results of α-ketoglutarate (αKG) from pαKG microparticles as determined by release measurements over the course of at least 30 days. FIG. 3E depicts ¹H NMR spectrum of pαKG polymer.

FIG. 4, comprising FIG. 4A through FIG. 4E, depicts representative data demonstrating that pαKG modulate adaptive immune responses in vitro. FIG. 4A depicts pαKG particles with rhodamine dye that were phagocytosed by DCs (green=cytosol, red=particles). FIG. 4B depicts representative data demonstrating that pαKG particles by themselves do not activate DCs (not significantly different than no treatment), but, in the presence of lipopolysaccharide (LPS), activate DCs. FIG. 4C depicts representative results demonstrating that pαKG particles in the presence of LPS induce significantly lower expression of IL-12 pro-inflammatory cytokine (normalized to LPS). FIG. 4D depicts representative results demonstrating that pαKG in the presence of LPS induce significantly higher levels of IL-(normalized to LPS). FIG. 4E depicts representative results demonstrating that pαKG substantially increased Treg/Th1 ratio in mixed lymphocyte reaction (n>4, ±stderror, * p<0.05).

FIG. 5, comprising FIG. 5A through FIG. 5D, depicts representative results demonstrating that pαKG microparticles effect function of dendritic cells by modulating their metabolism. FIG. 5A depicts representative data demonstrating that bone marrow-derived dendritic cells (BMDCs) are able to associate with pαKG microparticles (pαKG microparticle—red, cytosol—green, nucleus—blue; scale bar=70 μm). FIG. 5B depicts representative results demonstrating that pαKG MPs do not activate bone-marrow derived DCs as observed by frequency of MHCII⁺CD86⁺ in total CD11c⁺ population (n=6, avg±SEM, *—p<0.05). FIG. 5C depicts representative data demonstrating that pαKG microparticles prevent pro-inflammatory IL-12p70 cytokine production and at the same time do not decrease anti-inflammatory IL-10 production from BMDCs in vitro in the presence of LPS (n=3, avg±SEM, *—p<0.05). FIG. 5D depicts representative results of basal respiration, maximal respiration, and spare capacity of DCs demonstrating that the pαKG microparticles prevent oxygen consumption rate (OCR) of DCs. Moreover, decrease in non-glycolytic acidification and glycolysis demonstrate that extracellular acidification rate (ECAR) of DCs was significantly decreased by pαKG microparticles (n>15, avg±SEM, *—p<0.05).

FIG. 6, comprising FIG. 6A through FIG. 6E, depicts representative results demonstrating that pαKG microparticles modulate syngeneic and allogeneic adaptive immune responses in vitro by preventing decrease in Tregs and Th2 frequency. FIG. 6A depicts a schematic of flow plot analysis. (n>6, avg±SEM, *—p<0.05). FIG. 6B depicts representative data demonstrating that faster wound closure observed in pαKG group. FIG. 6C depicts representative data demonstrating that ultimate tensile strength is higher in pαKG group. FIG. 6D depicts representative data demonstrating that faster wound closure observed in pαKG group. FIG. 6E depicts representative results demonstrating that pαKG microparticles modulate syngeneic and allogeneic adaptive immune responses in vitro by preventing decrease in Tregs and Th2 frequency.

FIG. 7, comprising FIG. 7A through FIG. 7C, depicts representative results demonstrating that pαKG delivering modulate adaptive immune responses in vivo. FIG. 7A depicts representative data demonstrating that ultimate tensile strength is higher in pαKG group. FIG. 7B depicts representative data demonstrating that faster wound closure observed in pαKG group. FIG. 7C depicts representative data demonstrating that pαKG induced an immunosuppressive phenotype in the wound bed as observed by lower proliferating Th1, and Th17 and increased ratio of proliferating Tregs to Th1 (n=2, ±stdev).

FIG. 8, comprising FIG. 8A and FIG. 8B, depicts representative results demonstrating that pαKG microparticles modulate wound healing by increasing proliferation of anti-inflammatory Th2 frequency in draining lymph nodes. FIG. 8A depicts T-cells in skin. FIG. 8B depicts DCs in skin and T cells in draining lymph nodes.

FIG. 9, comprising FIG. 9A and FIG. 9B, depicts representative results demonstrating that PEGS microparticles are phagocytosed by dendritic cells. FIG. 9A depicts dendritic cells that are able to associate with PEGS microparticles (PEGS microparticles—red, cytosol—green, nucleus—blue; scale bar=70 μm). FIG. 9B depicts no treatment control of DCs.

FIG. 10 depicts scanning electron microscopy image of PEGS microparticles.

FIG. 11 depicts representative image of analyses of DCs using flow cytometry.

### DETAILED DESCRIPTION

The present invention provides compounds, particles (e.g., microparticles), and compositions that selectively and efficiently deliver various therapeutic agents, such as metabolites, to a cell. The present invention further relates to methods relating to the said compounds, particles (e.g., microparticles), and compositions for enhancing biological tissue growth (e.g. biological tissue regeneration and wound healing) in a subject. The compounds, particles (e.g., microparticles), and compositions of the present invention facilitate a decrease in the level of pro-inflammatory cytokine, an increase in the level of an anti-inflammatory cytokine, an increase in the level of a T regulatory cell, or any combination thereof. Thus, the present invention also relates, in part, to methods of treating or preventing diseases or disorders associated with increased level of a pro-inflammatory cytokine; decreased level of an anti-inflammatory cytokine; decreased level of a T regulatory cell; or any combination thereof in a subject in need thereof. The present invention additionally provides kits that find use in the practice of the methods of the invention.

### Definitions

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

The term “about” will be understood by persons of ordinary skill in the art and will vary to some extent depending on the context in which it is used. As used herein when referring to a measurable value such as an amount, a temporal duration, and the like, the term “about” is meant to encompass variations of ±20% or ±10%, more preferably ±5%, even more preferably ±1%, and still more preferably ±0.1% from the specified value, as such variations are appropriate to perform the disclosed methods.

As used herein, the term “alkyl,” by itself or as part of another substituent means, unless otherwise stated, a straight or branched chain hydrocarbon having the number of carbon atoms designated (i.e., C₁₋₆ means one to six carbon atoms) and includes straight, branched chain, or cyclic substituent groups. Examples include, but are not limited to, groups such as methyl, ethyl, n-propyl, isopropyl, n-butyl, t-butyl, isobutyl, sec-butyl, cyclohexyl, (cyclohexyl)methyl, cyclopropylmethyl, homologs and isomers of, for example, n-pentyl, n-hexyl, n-heptyl, n-octyl, and the like. The term “alkyl,” unless otherwise noted, is also meant to include those derivatives of alkyl defined in more detail below, such as “heteroalkyl”, “haloalkyl” and “homoalkyl”.

As used herein, the term “substituted alkyl” means alkyl, as defined above, substituted by one, two or three substituents selected from the group consisting of halogen, —OH, alkoxy, —NH₂, —N(CH₃)₂, —C(═O)OH, trifluoromethyl, —C═N, —C(═O)O(C₁-C₄)alkyl, —C(═O)NH₂, —SO₂NH₂, —C(═NH)NH₂, and —NO₂, preferably containing one or two substituents selected from halogen, —OH, alkoxy, —NH₂, trifluoromethyl, —N(CH₃)₂, and —C(═O)OH, more preferably selected from halogen, alkoxy and —OH. Examples of substituted alkyls include, but are not limited to, 2,2-difluoropropyl, 2-carboxycyclopentyl and 3-chloropropyl.

As used herein, the term “alkylene” by itself or as part of another molecule means a divalent radical derived from an alkane, as exemplified by (—CH₂—)ₙ. By way of example only, such groups include, but are not limited to, groups having 24 or fewer carbon atoms such as the structures —CH₂CH₂— and —CH₂CH₂CH₂CH₂—. The term “alkylene,” unless otherwise noted, is also meant to include those groups described below as “heteroalkylene.”

As used herein, the terms “alkoxy,” “alkylamino” and “alkylthio” are used in their conventional sense, and refer to alkyl groups linked to molecules via an oxygen atom, an amino group, a sulfur atom, respectively. As used herein, the term “alkoxy” employed alone or in combination with other terms means, unless otherwise stated, an alkyl group having the designated number of carbon atoms, as defined above, connected to the rest of the molecule via an oxygen atom, such as, for example, methoxy, ethoxy, 1-propoxy, 2-propoxy (isopropoxy) and the higher homologs and isomers. Preferred are (C₁-C₃) alkoxy, particularly ethoxy and methoxy.

As used herein, the term “halo” or “halogen” alone or as part of another substituent means, unless otherwise stated, a fluorine, chlorine, bromine, or iodine atom, preferably, fluorine, chlorine, or bromine, more preferably, fluorine or chlorine.

As used herein, the term “heteroalkyl” by itself or in combination with another term means, unless otherwise stated, a stable straight or branched chain alkyl group consisting of the stated number of carbon atoms and one or two heteroatoms selected from the group consisting of O, N, Si, P, and S, and wherein the nitrogen and sulfur atoms may be optionally oxidized and the nitrogen heteroatom may be optionally quaternized. The heteroatom(s) may be placed at any position of the heteroalkyl group, including between the rest of the heteroalkyl group and the fragment to which it is attached, as well as attached to the most distal carbon atom in the heteroalkyl group. Examples include: —O—CH₂—CH₂—CH₃, —CH₂—CH₂—CH₂—OH, —CH₂—CH₂—NH—CH₃, —CH₂—S—CH₂—CH₃, and —CH₂CH₂—S(═O)—CH₃. Up to two heteroatoms may be consecutive, such as, for example, —CH₂—NH—OCH₃, or —CH₂—CH₂—S—S—CH₃.

As used herein, the term “aromatic” refers to a carbocycle or heterocycle with one or more polyunsaturated rings and having aromatic character, i.e., having (4n+2) delocalized n (pi) electrons, where n is an integer.

As used herein, the term “aryl,” employed alone or in combination with other terms, means, unless otherwise stated, a carbocyclic aromatic system containing one or more rings (typically one, two or three rings) wherein such rings may be attached together in a pendent manner, such as a biphenyl, or may be fused, such as naphthalene. Examples include phenyl, anthracyl, and naphthyl. Preferred are phenyl and naphthyl, most preferred is phenyl.

As used herein, the term “heterocycle” or “heterocyclyl” or “heterocyclic” by itself or as part of another substituent means, unless otherwise stated, an unsubstituted or substituted, stable, mono- or multi-cyclic heterocyclic ring system that consists of carbon atoms and at least one heteroatom selected from the group consisting of N, O, and S, and wherein the nitrogen and sulfur heteroatoms may be optionally oxidized, and the nitrogen atom may be optionally quaternized. The heterocyclic system may be attached, unless otherwise stated, at any heteroatom or carbon atom that affords a stable structure. A heterocycle may be aromatic or non-aromatic in nature. In one embodiment, the heterocycle is a heteroaryl.

As used herein, the term “heteroaryl” or “heteroaromatic” refers to aryl groups which contain at least one heteroatom selected from N, O, Si, P, and S; wherein the nitrogen and sulfur atoms may be optionally oxidized, and the nitrogen atom(s) may be optionally quaternized. Heteroaryl groups may be substituted or unsubstituted. A heteroaryl group may be attached to the remainder of the molecule through a heteroatom. A polycyclic heteroaryl may include one or more rings that are partially saturated. Examples include tetrahydroquinoline, 2,3-dihydrobenzofuryl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl.

Examples of non-aromatic heterocycles include monocyclic groups such as aziridine, oxirane, thiirane, azetidine, oxetane, thietane, pyrrolidine, pyrroline, imidazoline, pyrazolidine, dioxolane, sulfolane, 2,3-dihydrofuran, 2,5-dihydrofuran, tetrahydrofuran, thiophane, piperidine, 1,2,3,6-tetrahydropyridine, 1,4-dihydropyridine, piperazine, morpholine, thiomorpholine, pyran, 2,3-dihydropyran, tetrahydropyran, 1,4-dioxane, 1,3-dioxane, homopiperazine, homopiperidine, 1,3-dioxepane, 4,7-dihydro-1,3-dioxepin and hexamethyleneoxide.

Examples of heteroaryl groups include pyridyl, pyrazinyl, pyrimidinyl (particularly 2- and 4-pyrimidinyl), pyridazinyl, thienyl, furyl, pyrrolyl (particularly 2-pyrrolyl), imidazolyl, thiazolyl, oxazolyl, pyrazolyl (particularly 3- and 5-pyrazolyl), isothiazolyl, 1,2,3-triazolyl, 1,2,4-triazolyl, 1,3,4-triazolyl, tetrazolyl, 1,2,3-thiadiazolyl, 1,2,3-oxadiazolyl, 1,3,4-thiadiazolyl and 1,3,4-oxadiazolyl.

Examples of polycyclic heterocycles include indolyl (particularly 3-, 4-, 5-, 6- and 7-indolyl), indolinyl, quinolyl, tetrahydroquinolyl, isoquinolyl (particularly 1- and 5-isoquinolyl), 1,2,3,4-tetrahydroisoquinolyl, cinnolinyl, quinoxalinyl (particularly 2- and 5-quinoxalinyl), quinazolinyl, phthalazinyl, 1,8-naphthyridinyl, 1,4-benzodioxanyl, coumarin, dihydrocoumarin, 1,5-naphthyridinyl, benzofuryl (particularly 3-, 4-, 5-, 6- and 7-benzofuryl), 2,3-dihydrobenzofuryl, 1,2-benzisoxazolyl, benzothienyl (particularly 3-, 4-, 5-, 6-, and 7-benzothienyl), benzoxazolyl, benzothiazolyl (particularly 2-benzothiazolyl and 5-benzothiazolyl), purinyl, benzimidazolyl (particularly 2-benzimidazolyl), benztriazolyl, thioxanthinyl, carbazolyl, carbolinyl, acridinyl, pyrrolizidinyl, and quinolizidinyl.

The aforementioned listing of heterocyclyl and heteroaryl moieties is intended to be representative and not limiting.

As used herein, the term “amino aryl” refers to an aryl moiety which contains an amino moiety. Such amino moieties may include, but are not limited to primary amines, secondary amines, tertiary amines, masked amines, or protected amines. Such tertiary amines, masked amines, or protected amines may be converted to primary amine or secondary amine moieties. Additionally, the amine moiety may include an amine-like moiety which has similar chemical characteristics as amine moieties, including but not limited to chemical reactivity.

As used herein, the term “substituted” means that an atom or group of atoms has replaced hydrogen as the substituent attached to another group. For aryl, aryl-(C₁-C₃)alkyl and heterocyclyl groups, the term “substituted” as applied to the rings of these groups refers to any level of substitution, namely mono-, di-, tri-, tetra-, or penta-substitution, where such substitution is permitted. The substituents are independently selected, and substitution may be at any chemically accessible position. In one embodiment, the substituents vary in number between one and four. In another embodiment, the substituents vary in number between one and three. In yet another embodiment, the substituents vary in number between one and two. In yet another embodiment, the substituents are independently selected from the group consisting of C₁₋₆ alkyl, —OH, C₁₋₆ alkoxy, halo, amino, acetamido and nitro. In yet another embodiment, the substituents are independently selected from the group consisting of C₁₋₆ alkyl, C₁₋₆ alkoxy, halo, acetamido, and nitro. As used herein, where a substituent is an alkyl or alkoxy group, the carbon chain may be branched, straight or cyclic, with straight being preferred.

As used herein, the term “protected,” as used herein, refers to the presence of a “protecting group” or moiety that prevents reaction of the chemically reactive functional group under certain reaction conditions. The protecting group will vary depending on the type of chemically reactive group being protected. By way of example only, (i) if the chemically reactive group is an amine or a hydrazide, the protecting group may be selected from tert-butyloxycarbonyl (t-Boc) and 9-fluorenylmethoxycarbonyl (Fmoc); (ii) if the chemically reactive group is a thiol, the protecting group may be orthopyridyldisulfide; and (iii) if the chemically reactive group is a carboxylic acid, such as butanoic or propionic acid, or a hydroxyl group, the protecting group may be benzyl or an alkyl group such as methyl, ethyl, or tert-butyl. Additionally, protecting groups include, but are not limited to, photolabile groups, such as Nvoc and MeNvoc, and other protecting groups known in the art. Other protecting groups are described in Greene and Wuts, Protective Groups in Organic Synthesis, 3rd Ed., John Wiley & Sons, New York, N.Y., 1999.

The term “derivative” refers to a small molecule that differs in structure from the reference molecule, but retains the essential properties of the reference molecule. A derivative may change its interaction with certain other molecules relative to the reference molecule. A derivative molecule may also include a salt, an adduct, tautomer, isomer, or other variant of the reference molecule.

The term “tautomers” are constitutional isomers of organic compounds that readily interconvert by a chemical process (tautomerization).

The term “isomers” or “stereoisomers” refer to compounds, which have identical chemical constitution, but differ with regard to the arrangement of the atoms or groups in space.

As used herein, the term “particle” refers to a number of particles, including, but not limited to, microparticles, nanoparticles, particle clusters, vesicles, capsule, ectosomes, micellar particles, lamellae shaped particles, polymersome particles, and other particles of various other small fabrications that are known to those in the art. The shapes and compositions of particles may be guided during condensation of atoms by selectively favoring growth of particular crystal facets to produce spheres, rods, wires, discs, cages, core-shell structures and many other shapes. The definitions and understandings of the entities falling within the scope of capsule are known to those of skill in the art. However, the following discussion is useful as a further understanding of some of these terms.

As used herein, the term “microparticle” refers to particles having a particle size on the micrometer scale, less than 1,000 micrometers (μm). For example, the microparticle may have a particle size up to about 50 μm. In another example, the microparticle may have a particle size up to about 10 μm. In another example, the microparticle may have a particle size up to about 6 μm. In another example, the microparticle may have a particle size up to about 1 μm. In another example, the microparticle may have a particle size up to about 0.1 μm. As used herein, “microparticle” refers to a number of microparticles, including, but not limited to, microparticle clusters, microvesicles, microcapsule, ectosomes, micellar microparticles, lamellae shaped microparticles, polymersome microparticles, and other micro-size particles of various other small fabrications that are known to those in the art. The shapes and compositions of microparticles may be guided during condensation of atoms by selectively favoring growth of particular crystal facets to produce spheres, rods, wires, discs, cages, core-shell structures and many other shapes. The definitions and understandings of the entities falling within the scope of microcapsule are known to those of skill in the art. However, the following discussion is useful as a further understanding of some of these terms.

For example, a “micellar microparticles” or “micelle”, a useful article in the employment of a general aspect of the present invention, can generally be thought of as a small—on the order of usually micrometers in diameter—aggregate of amphiphilic linear molecules having a polar, or hydrophilic end and an opposite non-polar, or hydrophobic end. These linear molecules can be comprised of simple molecules, or polymeric chains. A micellar microparticles or micelle can also be referred to as an aggregate of surfactant molecules dispersed in a liquid colloid. A typical micellar microparticles or micelle in aqueous solution can form an aggregate with the hydrophilic “head” regions in contact with surrounding solvent, and the sequestering of the hydrophobic tail regions in the micelle center. Other and similar definitions, descriptions and understandings of micelles are also known to those of skill in the art.

“Lamella” is a term whose definitions, descriptions and understandings are also known to those of skill in the art. In a very general sense, lamella or lamellae refers to plate-like, gill-shaped or other layered structures.

The definitions, descriptions and understandings of “microvesicle” are well known to those of skill in the art. For example, “microvesicle” can refer to a variety of small sac, sac-like or globular structures capable of containing fluid or other material therein.

“Pharmaceutically acceptable” refers to those properties and/or substances which are acceptable to the subject from a pharmacological/toxicological point of view and to the manufacturing pharmaceutical chemist from a physical/chemical point of view regarding composition, formulation, stability, subject acceptance and bioavailability. “Pharmaceutically acceptable carrier” refers to a medium that does not interfere with the effectiveness of the biological activity of the active ingredient(s) and is not toxic to the host to which it is administered.

As used herein, the term “pharmaceutically acceptable carrier” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound useful within the invention within or to the subject such that it may perform its intended function. Typically, such constructs are carried or transported from one organ, or portion of the body, to another organ, or portion of the body. Each carrier must be “acceptable” in the sense of being compatible with the other ingredients of the formulation, including the compound useful within the invention, and not injurious to the subject. Some examples of materials that may serve as pharmaceutically acceptable carriers include: sugars, such as lactose, glucose, and sucrose; starches, such as corn starch and potato starch; cellulose, and its derivatives, such as sodium carboxymethyl cellulose, ethyl cellulose, and cellulose acetate; powdered tragacanth; malt; gelatin; talc; excipients, such as cocoa butter and suppository waxes; oils, such as peanut oil, cottonseed oil, safflower oil, sesame oil, olive oil, corn oil, and soybean oil; glycols, such as propylene glycol; polyols, such as glycerin, sorbitol, mannitol, and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations. As used herein, “pharmaceutically acceptable carrier” also includes any and all coatings, antibacterial and antifungal agents, and absorption delaying agents, and the like that are compatible with the activity of the compound useful within the invention, and are physiologically acceptable to the subject. Supplementary active compounds may also be incorporated into the compositions. The “pharmaceutically acceptable carrier” may further include a pharmaceutically acceptable salt of the compound useful within the invention. Other additional ingredients that may be included in the pharmaceutical compositions used in the practice of the invention are known in the art.

The term “pharmaceutically acceptable salt” refers to any pharmaceutically acceptable salt, which upon administration to the subject is capable of providing (directly or indirectly) a compound as described herein. Such salts preferably are acid addition salts with physiologically acceptable organic or inorganic acids. Examples of the acid addition salts include mineral acid addition salts such as, for example, hydrochloride, hydrobromide, hydroiodide, sulphate, nitrate, phosphate, and organic acid addition salts such as, for example, acetate, trifluoroacetate, maleate, fumarate, citrate, oxalate, succinate, tartrate, malate, mandelate, methane sulphonate, and p-toluenesulphonate. Examples of the alkali addition salts include inorganic salts such as, for example, sodium, potassium, calcium and ammonium salts, and organic alkali salts such as, for example, ethylenediamine, ethanolamine, N,N-dialkylenethanolamine, triethanolamine, and basic amino acids salts. However, it will be appreciated that non-pharmaceutically acceptable salts also fall within the scope of the invention since those may be useful in the preparation of pharmaceutically acceptable salts. Procedures for salt formation are conventional in the art.

The term “solvate” in accordance with this invention should be understood as meaning any form of the active compound in accordance with the invention in which the said compound is bonded by a non-covalent bond to another molecule (normally a polar solvent), including especially hydrates and alcoholates.

As used herein, the term “pharmaceutical composition” refers to a mixture of at least one compound of the invention with other chemical components and entities, such as carriers, stabilizers, diluents, dispersing agents, suspending agents, thickening agents, and/or excipients. The pharmaceutical composition facilitates administration of the compound to an organism. Multiple techniques of administering a compound exist in the art including, but not limited to, intravenous, topical, intraperitoneal, intramuscular, oral, aerosol, parenteral, ophthalmic, pulmonary, and topical administration.

As used herein, the terms “therapeutic compound”, “therapeutic agent”, “drug”, “active pharmaceutical”, and “active pharmaceutical ingredient” are used interchangeably to refer to chemical entities that display certain pharmacological effects in a body and are administered for such purpose. Non-limiting examples of therapeutic agents include, but are not limited to, antibiotics, analgesics, vaccines, anticonvulsants; anti-diabetic agents, antifungal agents, antineoplastic agents, anti-parkinsonian agents, anti-rheumatic agents, appetite suppressants, biological response modifiers, cardiovascular agents, central nervous system stimulants, contraceptive agents, dietary supplements, vitamins, minerals, lipids, saccharides, metals, metabolites, amino acids (and precursors), nucleic acids and precursors, contrast agents, diagnostic agents, dopamine receptor agonists, erectile dysfunction agents, fertility agents, gastrointestinal agents, hormones, immunomodulators, antihypercalcemia agents, mast cell stabilizers, muscle relaxants, nutritional agents, ophthalmic agents, osteoporosis agents, psychotherapeutic agents, parasympathomimetic agents, parasympatholytic agents, respiratory agents, sedative hypnotic agents, skin and mucous membrane agents, smoking cessation agents, steroids, sympatholytic agents, urinary tract agents, uterine relaxants, vaginal agents, vasodilator, anti-hypertensive, hyperthyroids, anti-hyperthyroids, anti-asthmatics and vertigo agents. In certain embodiments, the one or more therapeutic agents are water-soluble, poorly water-soluble drug or a drug with a low, medium or high melting point. The therapeutic agents may be provided with or without a stabilizing salt or salts.

Some examples of active ingredients suitable for use in the pharmaceutical formulations and methods of the present invention include: hydrophilic, lipophilic, amphiphilic or hydrophobic, and that can be solubilized, dispersed, or partially solubilized and dispersed, on or about the microparticle cluster. The active agent-microparticle cluster combination may be coated further to encapsulate the agent-microparticle cluster combination and may be directed to a target by functionalizing the microparticle cluster with, e.g., aptamers and/or antibodies.

Alternatively, an active ingredient may also be provided separately from the solid pharmaceutical composition, such as for co-administration. Such active ingredients can be any compound or mixture of compounds having therapeutic or other value when administered to an animal, particularly to a mammal, such as drugs, nutrients, cosmeceuticals, nutraceuticals, diagnostic agents, nutritional agents, and the like. The active agents described herein may be found in their native state, however, they will generally be provided in the form of a salt. The active agents described herein include their isomers, analogs and derivatives.

The term “antibody”, as used herein, refers to an immunoglobulin molecule which is able to specifically bind to a specific epitope of an antigen. Antibodies can be intact immunoglobulins derived from natural sources, or from recombinant sources and can be immunoreactive portions of intact immunoglobulins. The antibodies in the present invention may exist in a variety of forms including, for example, polyclonal antibodies, monoclonal antibodies, multiple chain antibodies, intact immunoglobulins, synthetic antibodies, recombinant antibodies, intracellular antibodies (“intrabodies”), Fv, Fab, Fab′, F(ab)₂ and F(ab′)₂, as well as single chain antibodies (scFv), heavy chain antibodies, such as camelid antibodies, and humanized antibodies (Harlow et al., 1999, Using Antibodies: A Laboratory Manual, Cold Spring Harbor Laboratory Press, NY; Harlow et al., 1989, Antibodies: A Laboratory Manual, Cold Spring Harbor, N.Y.; Houston et al., 1988, Proc. Natl. Acad. Sci. USA 85:5879-5883; Bird et al., 1988, Science 242:423-426).

The term “antibody fragment” refers to at least one portion of an intact antibody, or recombinant variants thereof, and refers to the antigen binding domain, e.g., an antigenic determining variable region of an intact antibody, that is sufficient to confer recognition and specific binding of the antibody fragment to a target, such as an antigen.

By the term “synthetic antibody” as used herein, is meant an antibody which is generated using recombinant DNA technology, such as, for example, an antibody expressed by a bacteriophage. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using synthetic DNA or amino acid sequence technology which is available and well known in the art.

A “humanized antibody” refers to a type of engineered antibody having its CDRs derived from a non-human donor immunoglobulin, the remaining immunoglobulin-derived parts of the molecule being derived from one (or more) human immunoglobulin(s). In addition, framework support residues may be altered to preserve binding affinity (see, e.g., 1989, Queen et al., Proc. Natl. Acad Sci USA, 86:10029-10032; 1991, Hodgson et al., Bio/Technology, 9:421). A suitable human acceptor antibody may be one selected from a conventional database, e.g., the KABAT database, Los Alamos database, and Swiss Protein database, by homology to the nucleotide and amino acid sequences of the donor antibody. A human antibody characterized by a homology to the framework regions of the donor antibody (on an amino acid basis) may be suitable to provide a heavy chain constant region and/or a heavy chain variable framework region for insertion of the donor CDRs. A suitable acceptor antibody capable of donating light chain constant or variable framework regions may be selected in a similar manner. It should be noted that the acceptor antibody heavy and light chains are not required to originate from the same acceptor antibody. The prior art describes several ways of producing such humanized antibodies (see for example EP-A-0239400 and EP-A-054951).

A “chimeric antibody” refers to a type of engineered antibody which contains a naturally-occurring variable region (light chain and heavy chains) derived from a donor antibody in association with light and heavy chain constant regions derived from an acceptor antibody.

The term “donor antibody” refers to an antibody (monoclonal, and/or recombinant) which contributes the amino acid sequences of its variable regions, CDRs, or other functional fragments or analogs thereof to a first immunoglobulin partner, so as to provide the altered immunoglobulin coding region and resulting expressed altered antibody with the antigenic specificity and neutralizing activity characteristic of the donor antibody.

The term “acceptor antibody” refers to an antibody (monoclonal and/or recombinant) heterologous to the donor antibody, which contributes all (or any portion, but in some embodiments all) of the amino acid sequences encoding its heavy and/or light chain framework regions and/or its heavy and/or light chain constant regions to the first immunoglobulin partner. In certain embodiments a human antibody is the acceptor antibody.

By the term “recombinant antibody” as used herein, is meant an antibody which is generated using recombinant DNA technology, such as, for example, an antibody expressed by a bacteriophage or yeast expression system. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using recombinant DNA or amino acid sequence technology which is available and well known in the art.

An “antibody heavy chain,” as used herein, refers to the larger of the two types of polypeptide chains present in antibody molecules in their naturally occurring conformations, and which normally determines the class to which the antibody belongs.

An “antibody light chain,” as used herein, refers to the smaller of the two types of polypeptide chains present in antibody molecules in their naturally occurring conformations. Kappa (κ) and lambda (λ) light chains refer to the two major antibody light chain isotypes.

“CDRs” are defined as the complementarity determining region amino acid sequences of an antibody which are the hypervariable regions of immunoglobulin heavy and light chains. See, e.g., Kabat et al., Sequences of Proteins of Immunological Interest, 4th Ed., U.S. Department of Health and Human Services, National Institutes of Health (1987). There are three heavy chain and three light chain CDRs (or CDR regions) in the variable portion of an immunoglobulin. Thus, “CDRs” as used herein refers to all three heavy chain CDRs, or all three light chain CDRs (or both all heavy and all light chain CDRs, if appropriate). The structure and protein folding of the antibody may mean that other residues are considered part of the antigen binding region and would be understood to be so by a skilled person. See for example Chothia et al., (1989) Conformations of immunoglobulin hypervariable regions; Nature 342, p 877-883.

As used herein, the term “stabilizers” refers to either, or both, primary particle and/or secondary stabilizers, which may be polymers or other small molecules. Non-limiting examples of primary particle and/or secondary stabilizers for use with the present invention include, e.g., starch, modified starch, and starch derivatives, gums, including but not limited to polymers, polypeptides, albumin, amino acids, thiols, amines, carboxylic acid and combinations or derivatives thereof. Other examples include xanthan gum, alginic acid, other alginates, benitoniite, veegum, agar, guar, locust bean gum, gum arabic, quince psyllium, flax seed, okra gum, arabinoglactin, pectin, tragacanth, scleroglucan, dextran, amylose, amylopectin, dextrin, etc., cross-linked polyvinylpyrrolidone, ion-exchange resins, potassium polymethacrylate, carrageenan (and derivatives), gum karaya and biosynthetic gum. Other examples of useful primary particle and/or secondary stabilizers include polymers such as: polycarbonates (linear polyesters of carbonic acid); microporous materials (bisphenol, a microporous poly(vinylchloride), micro-porous polyamides, microporous modacrylic copolymers, microporous styrene-acrylic and its copolymers); porous polysulfones, halogenated poly(vinylidene), polychloroethers, acetal polymers, polyesters prepared by esterification of a dicarboxylic acid or anhydride with an alkylene polyol, poly(alkylenesulfides), phenolics, polyesters, asymmetric porous polymers, cross-linked olefin polymers, hydrophilic microporous homopolymers, copolymers or interpolymers having a reduced bulk density, and other similar materials, poly(urethane), cross-linked chain-extended poly(urethane), poly(mides), poly(benzimidazoles), collodion, regenerated proteins, semi-solid cross-linked poly(vinylpyrrolidone).

As used herein, the terms “targeting domain”, “targeting moiety”, or “targeting group” are used interchangeably and refer to all molecules capable of specifically binding to a particular target molecule and forming a bound complex as described above. Thus, the ligand and its corresponding target molecule form a specific binding pair.

As used herein, the term “specific binding” refers to that binding which occurs between such paired species as enzyme/substrate, receptor/agonist, antibody/antigen, and lectin/carbohydrate which may be mediated by covalent or non-covalent interactions or a combination of covalent and non-covalent interactions. When the interaction of the two species produces a non-covalently bound complex, the binding which occurs is typically electrostatic, hydrogen-bonding, or the result of lipophilic interactions. Accordingly, “specific binding” occurs between a paired species where there is interaction between the two which produces a bound complex having the characteristics of an antibody/antigen or enzyme/substrate interaction. In particular, the specific binding is characterized by the binding of one member of a pair to a particular species and to no other species within the family of compounds to which the corresponding member of the binding member belongs. Thus, for example, an antibody preferably binds to a single epitope and to no other epitope within the family of proteins.

The term “specifically binds”, as used herein with respect to an antibody, is meant for an antibody which recognizes a specific antigen, but does not substantially recognize or bind other molecules in a sample. For example, an antibody that specifically binds to an antigen from one species may also bind to that antigen from one or more species. But, such cross-species reactivity does not itself alter the classification of an antibody as specific. In another example, an antibody that specifically binds to an antigen may also bind to different allelic forms of the antigen. However, such cross reactivity does not itself alter the classification of an antibody as specific. In some instances, the terms “specific binding” or “specifically binding,” can be used in reference to the interaction of an antibody, a protein, or a peptide with a second chemical species, to mean that the interaction is dependent upon the presence of a particular structure (e.g., an antigenic determinant or epitope) on the chemical species; for example, an antibody recognizes and binds to a specific protein structure rather than to proteins generally. If an antibody is specific for epitope “A”, the presence of a molecule containing epitope A (or free, unlabeled A), in a reaction containing labeled “A” and the antibody, will reduce the amount of labeled A bound to the antibody.

As used herein, the terms “peptide”, “polypeptide”, and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or any combination thereof.

“Isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

The term “nutritional composition” may be a food product intended for human consumption, for example, a beverage, a drink, a bar, a snack, an ice cream, a dairy product, for example a chilled or a shelf-stable dairy product, a fermented dairy product, a drink, for example a milk-based drink, an infant formula, a growing-up milk, a confectionery product, a chocolate, a cereal product such as a breakfast cereal, a sauce, a soup, an instant drink, a frozen product intended for consumption after heating in a microwave or an oven, a ready-to-eat product, a fast food or a nutritional formula.

As used herein, the term “T cell” refers to a lymphocyte (e.g., white blood cell) that functions in cell-mediated immunity. In some embodiments, the presence of a T cell receptor (TCR) on the cell surface distinguishes T cells from other lymphocytes. As is known in the art, T cells typically do not present antigens, and rely on other lymphocytes (e.g., natural killer cells and B cells) to aid in antigen presentation. Types of T cells include: T helper cells (TH cells), Memory T cells (Tcm, Tem, or Temra), Regulatory T cells (Treg), Cytotoxic T cells (CTLs), Natural killer T cells (NK cells), gamma delta T cells, and Mucosal associated invariant T cells (MAIT).

The terms “patient,” “subject,” “individual,” and the like are used interchangeably herein, and refer to any animal, or cells thereof whether in vitro or in situ, amenable to the methods described herein. In certain non-limiting embodiments, the patient, subject or individual is a human. In various embodiments, the subject is a human subject, and may be of any race, ethnicity, sex, and age.

The terms “effective amount” and “pharmaceutically effective amount” refer to a sufficient amount of an agent to provide the desired biological result. That result can be reduction and/or alleviation of a sign, symptom, or cause of a disease or disorder, or any other desired alteration of a biological system. An appropriate effective amount in any individual case may be determined by one of ordinary skill in the art using routine experimentation.

A “therapeutically effective amount” refers to that amount which provides a therapeutic effect for a given condition and administration regimen. In particular, “therapeutically effective amount” means an amount that is effective to prevent, alleviate or ameliorate symptoms of the disease or prolong the survival of the subject being treated, which may be a human or non-human animal. Determination of a therapeutically effective amount is within the skill of the person skilled in the art.

A “therapeutic” treatment is a treatment administered to a subject who exhibits signs or symptoms of a disease or disorder, for the purpose of diminishing or eliminating those signs or symptoms.

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

As used herein, “treating a disease or disorder” means reducing the severity and/or frequency with which a sign or symptom of the disease or disorder is experienced by a subject.

A disease or disorder is “alleviated” if the severity of a sign or symptom of the disease or disorder, the frequency with which such a sign or symptom is experienced by a subject, or both, is reduced.

“Instructional material”, as that term is used herein, includes a publication, a recording, a diagram, or any other medium of expression which can be used to communicate the usefulness of the nucleic acid, peptide, and/or compound of the invention in the kit for identifying, diagnosing or alleviating or treating the various diseases or disorders recited herein. Optionally, or alternately, the instructional material may describe one or more methods of identifying, diagnosing or alleviating the diseases or disorders in a cell or a tissue of a subject. The instructional material of the kit may, for example, be affixed to a container that contains one or more components of the invention or be shipped together with a container that contains the one or more components of the invention. Alternatively, the instructional material may be shipped separately from the container with the intention that the recipient uses the instructional material and the components cooperatively.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range, such as from 1 to 6, should be considered to have specifically disclosed subranges, such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### DESCRIPTION

The present invention provides compounds, particles (e.g., microparticles), and compositions that deliver various therapeutic agents, such as metabolites, to a cell. The present invention further relates to methods relating to the said compounds, particles (e.g., microparticles), and compositions for enhancing biological tissue growth (e.g. biological tissue regeneration and wound healing) in a subject. The compounds, particles (e.g., microparticles), and compositions of the present invention also facilitate a decrease in the level of pro-inflammatory cytokine, an increase in the level of an anti-inflammatory cytokine, an increase in the level of a T regulatory cell, or any combination thereof. Thus, the present invention also relates, in part, to methods of treating or preventing diseases or disorders associated with increased level of a pro-inflammatory cytokine; decreased level of an anti-inflammatory cytokine; decreased level of a T regulatory cell; or any combination thereof in a subject in need thereof. The present invention additionally provides kits that find use in the practice of the methods of the invention.

### Polymers

In one aspect, the invention provides polymers comprising a metabolite. In one embodiment, the metabolite modulates the function of an immune cell. In one embodiment, the polymer comprises alpha-ketoglutarate (αKG), citrate, isocitrate, succinate, fumarate, malate, spermidine, itaconate, oxaloacetate, or any combination thereof. In one embodiment, the polymer comprises αKG.

In one aspect, the invention provides a compound or salt thereof comprising the structure of Formula (I)

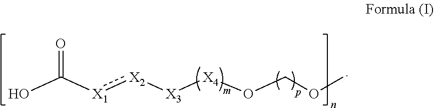

In some embodiments, each occurrence of X₁ is independently C═R₁, CR₂, or CR₃R₄. In some embodiments, each occurrence of X₂ is independently C═R₁, CR₂, or CR₃R₄. In some embodiments, each occurrence of X₃ is C═R₁ or CR₃R₄. In some embodiments, each occurrence of X₄ is independently C═R₁ or CR₃R₄.

In some embodiments, the bond between X₁ and X₂ is a single bond or a double bond. In some embodiments, when the bond between X₁ and X₂ is a single bond, X₁ and X₂ are each independently C═R₁ or CR₃R₄. In one embodiment, when the bond between X₁ and X₂ is a double bond, X₁ and X₂ are each C—R₂.

In some embodiments, R₁ is O, NH, or S. In one embodiment R₁ is O.

In some embodiments, R₂ is hydrogen, hydroxyl, carboxyl, alkyl, cycloalkyl, heterocycloalkyl, aryl, or heteroaryl. In one embodiment, R₂ is hydrogen. In one embodiment, R₂ is hydroxyl.

In some embodiments, R₃ is hydrogen, hydroxyl, carboxyl, alkyl, cycloalkyl, heterocycloalkyl, aryl, or heteroaryl. In one embodiment, R₃ is hydrogen. In one embodiment, R₃ is hydroxyl. In one embodiment, R₃ is carboxyl.

In some embodiments, R₄ is hydrogen, hydroxyl, carboxyl, alkyl, cycloalkyl, heterocycloalkyl, aryl, or heteroaryl. In one embodiment, R₄ is hydrogen. In one embodiment, R₄ is hydroxyl. In one embodiment, R₄ is carboxyl.

In some embodiments, m is an integer represented by 0, 1, 2, or 5.

In some embodiments, p is an integer from 1 to 50. In some embodiments, p is an integer from 1 to 15. In some embodiments, p is an integer from 1 to 10. For example, in one embodiment, p is an integer represented by 9. In another embodiment, p is an integer represented by 10.

In some embodiments, n is an integer from 1 to 1000.

In various embodiments, the compound or salt thereof comprising the structure of Formula (I) is a compound comprising the structure of Formula (II)-Formula (XI):

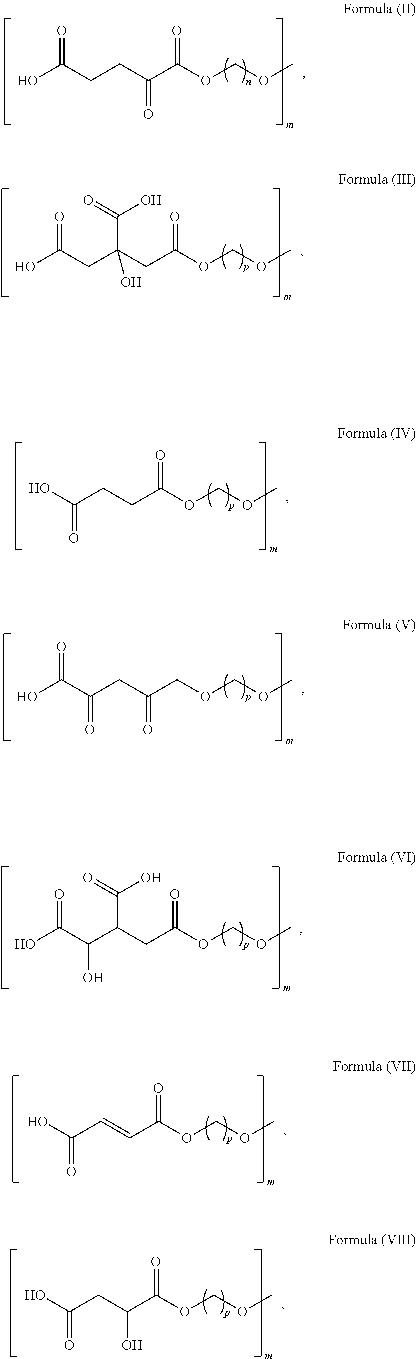

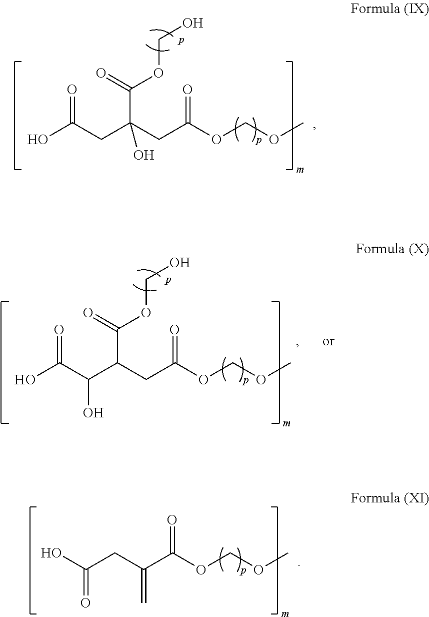

In some embodiments, in Formulae (II)-(XI), p is an integer from 1 to 50. In some embodiments, p is an integer from 1 to 15. In some embodiments, p is an integer from 1 to 10.

For example, in one embodiment, p is an integer represented by 9. In another embodiment, p is an integer represented by 10.

### Particles/Microparticles

In one aspect, the present invention also provides a particle comprising at least one compound described herein. For example, in one embodiment, one or more compounds of Formula (I) form the particle. Thus, in various embodiments, the present invention discloses a particle comprising at least one compound or salt thereof comprising the structure of Formula (I).

In various embodiments, the particle is a microparticle. In some embodiments, the microparticle has an average size (i.e., average diameter of the microparticle) of about 0.01 μm to about 1000 μm. For example, in one embodiment, the microparticle has an average size (i.e., average diameter of the microparticle) of about 0.01 μm. In another embodiment, the microparticle has an average size (i.e., average diameter of the microparticle) of about 10 μm.

In various embodiments, the microparticle is any type of microparticle, including, but not limited to, a microparticle cluster, microvesicle, microcarrier, microcapsule, ectosomes, micellar microparticles, lamellae shaped microparticles, polymersome microparticles, polymer vesicle, and micro-size particles of various other small fabrications that are known to those in the art.

In some embodiments, the particle is a biodegradable particle. For example, in one embodiment, the microparticle is biodegradable microcapsule. In another embodiment, the microparticle is a biodegradable polymer vesicle.

In various embodiments, the particle (e.g., microparticle) comprises at least one therapeutic agent. In some embodiments, the particle (e.g., microparticle) encapsulates at least one therapeutic agent. In one embodiment, the particle (e.g., microparticle) is bound to the therapeutic agent.

Examples of such therapeutic agents include, but are not limited to, one or more drugs, metabolites, proteins, amino acids, peptides, antibodies, medical imaging agents, therapeutic moieties, one or more non-therapeutic moieties or a combination to target cancer or atherosclerosis, selected from folic acid, peptides, proteins, aptamers, antibodies, siRNA, poorly water soluble drugs, anti-cancer drugs, antibiotics, analgesics, vaccines, anticonvulsants; anti-diabetic agents, antifungal agents, antineoplastic agents, anti-parkinsonian agents, anti-rheumatic agents, appetite suppressants, biological response modifiers, cardiovascular agents, central nervous system stimulants, contraceptive agents, dietary supplements, vitamins, minerals, lipids, saccharides, metals, amino acids (and precursors), nucleic acids and precursors, contrast agents, diagnostic agents, dopamine receptor agonists, erectile dysfunction agents, fertility agents, gastrointestinal agents, hormones, immunomodulators, antihypercalcemia agents, mast cell stabilizers, muscle relaxants, nutritional agents, ophthalmic agents, osteoporosis agents, psychotherapeutic agents, parasympathomimetic agents, parasympatholytic agents, respiratory agents, sedative hypnotic agents, skin and mucous membrane agents, smoking cessation agents, steroids, sympatholytic agents, urinary tract agents, uterine relaxants, vaginal agents, vasodilator, anti-hypertensive, hyperthyroids, anti-hyperthyroids, anti-asthmatics and vertigo agents, or any combinations thereof.

In one embodiment, the therapeutic agent is one or more non-therapeutic moieties. In some embodiments, the particle (e.g., microparticle) comprises one or more therapeutic moieties, one or more non-therapeutic moieties, or any combination thereof. In some embodiments, the composition comprises folic acid, peptides, proteins, aptamers, antibodies, small RNA molecules, miRNA, shRNA, siRNA, poorly water-soluble therapeutic agents, anti-cancer agents, or any combinations thereof.

In one aspect of the invention, the particle (e.g., microparticle) releases at least one therapeutic agent. In some embodiments, the particle (e.g., microparticle) releases at least one therapeutic agent inside or outside the cell. In some embodiments, the particle (e.g., microparticle) decomposes or degrades to release at least one therapeutic agent.

In another aspect of the invention, the particle (e.g., microparticle) releases at least one metabolite. In some embodiments, the particle (e.g., microparticle) decomposes or degrades to release at least one metabolite. Thus, in various embodiments, the therapeutic agent is a metabolite. In some embodiments, the metabolite is α-ketoglutarate (αKG), citrate, isocitrate, succinate, fumarate, malate, oxaloacetate, spermidine, itaconate, or any combination thereof.

In one embodiment, the particle (e.g., microparticle) is phagocytosed by a cell. Examples of such cells include, but are not limited to, antigen-presenting cells (APC), accessory cell, dendritic cells, T cells, B cells, and macrophages.

In one embodiment, the particle (e.g., microparticle) further comprises a targeting domain. In one aspect, the particle (e.g., microparticle) further comprises a targeting domain attached to the surface of the particle (e.g., microparticle). In some embodiments, the targeting domain is bound to an exterior surface of the particle (e.g., microparticle) and recognizes a particular site of interest in a subject. In one embodiment, the targeting domain binds to at least one associated with a disease or a disorder. In various embodiments, the targeting domain is an antibody, an antibody fragment, a peptide sequence, aptamer, folate, a ligand, a gene component, or any combination thereof. Examples of targeting domains include, but are not limited to antibodies, lymphokines, cytokines, receptor proteins such as CD4 and CD8, solubilized receptor proteins such as soluble CD4, hormones, growth factors, peptidomimetics, synthetic ligands, and the like which specifically bind desired target cells, and nucleic acids which bind corresponding nucleic acids through base pair complementarity. Targeting domains of particular interest include peptidomimetics, peptides, antibodies (e.g., monoclonal antibodies, polyclonal antibodies, recombinant antibodies, human antibodies, humanized antibodies, etc.) and antibody fragments (e.g., the Fab′ fragment).

Methods of making and using antibodies are well known in the art. For example, polyclonal antibodies useful in the present invention are generated by immunizing rabbits according to standard immunological techniques well-known in the art. Such techniques include immunizing an animal with a chimeric protein comprising a portion of another protein such as a maltose binding protein or glutathione (GSH) tag polypeptide portion, and/or a moiety such that the antigenic protein of interest is rendered immunogenic (e.g., an antigen of interest conjugated with keyhole limpet hemocyanin, KLH) and a portion comprising the respective antigenic protein amino acid residues.

However, the invention should not be construed as being limited solely to methods and compositions including these antibodies or to these portions of the antigens. Rather, the invention should be construed to include other antibodies, as that term is defined elsewhere herein, to antigens, or portions thereof. Further, the present invention should be construed to encompass antibodies, inter alia, which bind to the specific antigens of interest.

One skilled in the art would appreciate, based upon the disclosure provided herein, that the antibody can specifically bind with any portion of an antigen target, which can be used to generate antibodies specific therefor. However, the present invention is not limited to using the full-length protein as an immunogen. Rather, the present invention includes using an immunogenic portion of the protein to produce an antibody that specifically binds with a specific antigen. That is, the invention includes immunizing an animal using an immunogenic portion, or antigenic determinant, of the antigen.

The antibodies can be produced by immunizing an animal such as, but not limited to, a rabbit, a mouse or a camel, with an antigenic protein of the invention, or a portion thereof, by immunizing an animal using a protein comprising at least a portion of the antigen, or a fusion protein including a tag polypeptide portion comprising, for example, a maltose binding protein tag polypeptide portion, covalently linked with a portion comprising the appropriate amino acid residues. One skilled in the art would appreciate, based upon the disclosure provided herein, that smaller fragments of these proteins can also be used to produce antibodies that specifically bind the antigen of interest.

Once armed with the sequence of a specific antigen of interest and the detailed analysis localizing the various conserved and non-conserved domains of the protein, the skilled artisan would understand, based upon the disclosure provided herein, how to obtain antibodies specific for the various portions of the antigen using methods well-known in the art or to be developed.

Further, the skilled artisan, based upon the disclosure provided herein, would appreciate that using a non-conserved immunogenic portion can produce antibodies specific for the non-conserved region thereby producing antibodies that do not cross-react with other proteins which can share one or more conserved portions. Thus, one skilled in the art would appreciate, based upon the disclosure provided herein, that the non-conserved regions of an antigen of interest can be used to produce antibodies that are specific only for that antigen and do not cross-react non-specifically with other proteins.

The invention encompasses monoclonal, synthetic antibodies, and the like. One skilled in the art would understand, based upon the disclosure provided herein, that the crucial feature of the antibody of the invention is that the antibody bind specifically with an antigen of interest. That is, the antibody of the invention recognizes an antigen of interest or a fragment thereof (e.g., an immunogenic portion or antigenic determinant thereof).

The skilled artisan would appreciate, based upon the disclosure provided herein, that present invention includes use of a single antibody recognizing a single antigenic epitope but that the invention is not limited to use of a single antibody. Instead, the invention encompasses use of at least one antibody where the antibodies can be directed to the same or different antigenic protein epitopes.

The generation of polyclonal antibodies is accomplished by inoculating the desired animal with the antigen and isolating antibodies which specifically bind the antigen therefrom using standard antibody production methods such as those described in, for example, Harlow et al. (1988, In: Antibodies, A Laboratory Manual, Cold Spring Harbor, N.Y.).

Monoclonal antibodies directed against full length or peptide fragments of a protein or peptide may be prepared using any well-known monoclonal antibody preparation procedures, such as those described, for example, in Harlow et al. (1988, In: Antibodies, A Laboratory Manual, Cold Spring Harbor, N.Y.) and in Tuszynski et al. (1988, Blood, 72:109-115). Quantities of the desired peptide may also be synthesized using chemical synthesis technology. Alternatively, DNA encoding the desired peptide may be cloned and expressed from an appropriate promoter sequence in cells suitable for the generation of large quantities of peptide. Monoclonal antibodies directed against the peptide are generated from mice immunized with the peptide using standard procedures as referenced herein.

Nucleic acid encoding the monoclonal antibody obtained using the procedures described herein may be cloned and sequenced using technology which is available in the art, and is described, for example, in Wright et al. (1992, Critical Rev. Immunol. 12:125-168), and the references cited therein. Further, the antibody of the invention may be “humanized” using the technology described in, for example, Wright et al., and in the references cited therein, and in Gu et al. (1997, Thrombosis and Hematocyst 77:755-759), and other methods of humanizing antibodies well-known in the art or to be developed.

In some embodiments, a non-human antibody is humanized, where specific sequences or regions of the antibody are modified to increase similarity to an antibody naturally produced in a human or fragment thereof. A humanized antibody can be produced using a variety of techniques known in the art, including but not limited to, CDR-grafting (see, e.g., European Patent No. EP 239,400; International Publication No. WO 91/09967; and U.S. Pat. Nos. 5,225,539, 5,530,101, and 5,585,089), veneering or resurfacing (see, e.g., European Patent Nos. EP 592,106 and EP 519,596; Padlan, 1991, Molecular Immunology, 28(4/5):489-498; Studnicka et al., 1994, Protein Engineering, 7(6):805-814; and Roguska et al., 1994, PNAS, 91:969-973), chain shuffling (see, e.g., U.S. Pat. No. 5,565,332), and techniques disclosed in, e.g., U.S. Patent Application Publication No. US2005/0042664, U.S. Patent Application Publication No. US2005/0048617, U.S. Pat. Nos. 6,407,213, 5,766,886, International Publication No. WO 9317105, Tan et al., J. Immunol., 169:1119-25 (2002), Caldas et al., Protein Eng., 13(5):353-60 (2000), Morea et al., Methods, 20(3):267-79 (2000), Baca et al., J. Biol. Chem., 272(16):10678-84 (1997), Roguska et al., Protein Eng., 9(10):895-904 (1996), Couto et al., Cancer Res., 55 (23 Supp):5973s-5977s (1995), Couto et al., Cancer Res., 55(8):1717-22 (1995), Sandhu J S, Gene, 150(2):409-10 (1994), and Pedersen et al., J. Mol. Biol., 235(3):959-73 (1994). Often, framework residues in the framework regions will be substituted with the corresponding residue from the CDR donor antibody to alter, for example improve, antigen binding. These framework substitutions are identified by methods well-known in the art, e.g., by modeling of the interactions of the CDR and framework residues to identify framework residues important for antigen binding and sequence comparison to identify unusual framework residues at particular positions. (See, e.g., Queen et al., U.S. Pat. No. 5,585,089; and Riechmann et al., 1988, Nature, 332:323.)

In one embodiment, the antibody fragment provided herein is a single chain variable fragment (scFv). In various embodiments, the antibodies of the invention may exist in a variety of other forms including, for example, Fv, Fab, and (Fab′) 2, as well as bi-functional (i.e. bi-specific) hybrid antibodies (e.g., Lanzavecchia et al., Eur. J. Immunol. 17, 105 (1987)). In some embodiments, the antibodies and fragments thereof of the invention bind a cell bearing antigen, TCR, and/or BCR with wild-type or enhanced affinity. In some embodiments, the antibodies and fragments thereof of the invention bind a T cell bearing TCR with wild-type or enhanced affinity. In some embodiments, the antibodies and fragments thereof of the invention bind a B cell bearing BCR with wild-type or enhanced affinity. In various embodiments, a human scFv may also be derived from a yeast display library.

ScFvs can be prepared according to method known in the art (see, for example, Bird et al., (1988) Science 242:423-426 and Huston et al., (1988) Proc. Natl. Acad. Sci. USA 85:5879-5883). ScFv molecules can be produced by linking VH and VL regions together using flexible polypeptide linkers. The scFv molecules comprise flexible polypeptide linker (e.g., a Ser-Gly linker) with an optimized length and/or amino acid composition. The flexible polypeptide linker length can greatly affect how the variable regions of an scFv fold and interact. In fact, if a short polypeptide linker is employed (e.g., between 5-10 amino acids, intrachain folding is prevented. Interchain folding is also required to bring the two variable regions together to form a functional epitope binding site. For examples of linker orientation and size see, e.g., Hollinger et al. 1993 Proc Natl Acad. Sci. U.S.A. 90:6444-6448, U.S. Patent Application Publication Nos. 2005/0100543, 2005/0175606, 2007/0014794, and PCT publication Nos. WO2006/020258 and WO2007/024715.

The scFv can comprise a polypeptide linker sequence of at least 1, 2, 3, 4, 5, 6, 7, 8, 9, 10, 11, 12, 13, 14, 15, 16, 17, 18, 19, 20, 25, 30, 35, 40, 45, 50, or more amino acid residues between its VL and VH regions. The flexible polypeptide linker sequence may comprise any naturally occurring amino acid. In some embodiments, the flexible polypeptide linker sequence comprises amino acids glycine and serine. In another embodiment, the flexible polypeptide linker sequence comprises sets of glycine and serine repeats such as (Gly4Ser)n, where n is a positive integer equal to or greater than 1. In one embodiment, the flexible polypeptide linkers include, but are not limited to, (Gly4Ser)4 or (Gly4Ser)3. Variation in the flexible polypeptide linker length may retain or enhance activity, giving rise to superior efficacy in activity studies.

In one embodiment, the targeting domain is bound directly to the particle (e.g., microparticle). In one embodiment, the targeting domain is bound directly to the surface of the particle (e.g., microparticle). In one embodiment, the targeting domain is bound to the particle (e.g., microparticle) using a linking molecule. In one embodiment, the targeting domain is bound to the surface of the particle (e.g., microparticle) using a linking molecule. The linking molecules useful in the compositions and methods of the present disclosure may be any molecule capable of binding to both the particle (e.g., microparticle) and the targeting domains used in the compositions and methods of the present disclosure. In certain embodiments, the linking molecule may be a hydrophilic polymer. Examples of linking molecules include, but are not limited to, poly(ethylene glycol) and its derivatives, dithiol compounds, dithiol compounds with hydrazide and/or carboxylic functionality, or single thiols and/or amines or their derivatives.

In certain embodiments, the linking molecule and the targeting domain may be bound by one or more covalent bonds. In certain embodiments, the linking molecule, in addition to linking the targeting domain and the particle (e.g., microparticle), may impart certain benefits upon the compositions of the present disclosure, including, but not limited to, improved hydrophilicity and stability in solution, reduced immunogenic responses upon introduction of the compositions of the present disclosure into a subject, increased circulation time of the compositions of the present disclosure when introduced into the bloodstream of a subject. The choice of a linking molecule may depend upon, among other things, the targeting domain chosen and the subject into which the compositions of the present invention are to be introduced. One of ordinary skill in the art, with the benefit of this disclosure, will recognize additional suitable linking molecules. Such linking molecules are considered to be within the spirit of the present disclosure.

In certain embodiments, the targeting domain may recognize a particular ligand or receptor present in a desired cell and/or tissue type when introduced into a subject. In certain embodiments, the targeting domain may be an antibody that recognizes such a particular ligand or receptor. The use of antibody fragments may also be suitable in the compositions of the present disclosure. The choice of a targeting domain may depend upon, among other things, the cell and/or tissue type into which an at least partial increase in uptake of the compositions of the present disclosure is desired, as well as particular ligand(s) present in such cell and/or tissue types.

In certain embodiments, the targeting domain may be chosen, among other things, to at least partially increase the uptake of the particle (e.g., microparticle) of the present disclosure into a desired cell and/or tissue type when introduced into a subject.

In some embodiments, the suitable targeting domain may be a peptide sequence, DNA fragment, aptamer, RNA, folate, polymer, etc. One of ordinary skill in the art, with the benefit of this disclosure, will recognize other targeting domains that may be useful in the compositions of the present disclosure. Such targeting domains are considered to be within the spirit of the present disclosure.

To obtain additional selectivity, the particle (e.g., microparticle) may be passively or actively targeted to regions of interest, such as organs, vessels, sites of disease, wounds, or a specific organism in a subject. In active targeting, the particle (e.g., microparticle) may be attached to biological recognition agents to allow them to accumulate in or to be selectively retained by or to be slowly eliminated from certain parts of the body, such as specific organs, parts of organs, bodily structures and disease structures and lesions. Active targeting is defined as a modification of biodistribution using chemical groups that will associate with species present in the desired tissue or organism to effectively decrease the rate of loss of particle (e.g., microparticle) from the specific tissue or organism.

Active targeting of the particle (e.g., microparticle) can be considered as localization through modification of biodistribution of the particle (e.g., microparticle) by means of a targeting domain that is attached to or incorporated into the particle (e.g., microparticle). The targeting domain can associate or bind with one or more receptor species present in the tissue or organism of interest. This binding will effectively decrease the rate of loss of particle (e.g., microparticle) from the specific tissue or organism of interest. In such cases, the particle (e.g., microparticle) can be modified synthetically to incorporate the targeting domain. Targeted particle (e.g., microparticle) can localize because of binding between the ligand and the targeted receptor. Alternatively, the particle (e.g., microparticle) can distribute by passive biodistribution, i.e., by passive targeting, into diseased tissues of interest such as wounds. Thus, even without synthetic manipulation to incorporate a targeting domain that can bind to a receptor site, passively targeted contrast agents can accumulate in a diseased tissue or in specific locations in the subject, such as the skin. The present invention comprises use of a particle (e.g., microparticle) that is linked to a targeting domain that has an affinity for binding to a receptor. Preferably the receptor is located on the surface of a diseased cell or wounded tissue in a human or animal subject.

In some embodiments, the particle (e.g., microparticle) further comprises a biocompatible metal. Examples of biocompatible metals include, but are not limited to, copper, iron oxide, cobalt and noble metals, such as gold and/or silver. One of ordinary skill in the art will be able to select of a suitable type of particle (e.g., microparticle) taking into consideration at least the type of imaging and/or therapy to be performed.

The present invention also provides various compositions comprising the particles (e.g., microparticles) of the present invention. In some embodiments, the composition comprises a microparticle cluster, microcarrier, microparticle cluster composition, microcarrier composition, contrast agent composition, or any combination thereof. In one embodiment, the microparticle cluster composition is a biodegradable microparticle cluster composition. In one embodiment, the microparticle cluster composition is a medical biodegradable microparticle cluster composition.

In various aspects, the composition comprises: one or more particles (e.g., microparticles) of the present invention and one or more stabilizers. In various embodiments, the stabilizer to particle (e.g., microparticle) weight ratio is less than 50%. In one embodiment, the stabilizer comprises a biocompatible polymer. Examples of stabilizers include, but are not limited to, biocompatible polymer, a biodegradable polymer, a multifunctional linker, starch, modified starch, and starch derivatives, gums, including but not limited to polymers, polypeptides, albumin, amino acids, thiols, amines, carboxylic acid and combinations or derivatives thereof, citric acid, xanthan gum, alginic acid, other alginates, benitoniite, veegum, agar, guar, locust bean gum, gum arabic, quince psyllium, flax seed, okra gum, arabinoglactin, pectin, tragacanth, scleroglucan, dextran, amylose, amylopectin, dextrin, etc., cross-linked polyvinylpyrrolidone, ion-exchange resins, potassium polymethacrylate, carrageenan (and derivatives), gum karaya and biosynthetic gum, polycarbonates (linear polyesters of carbonic acid); microporous materials (bisphenol, a microporous poly(vinylchloride), micro-porous polyamides, microporous modacrylic copolymers, microporous styrene-acrylic and its copolymers); porous polysulfones, halogenated poly(vinylidene), polychloroethers, acetal polymers, polyesters prepared by esterification of a dicarboxylic acid or anhydride with an alkylene polyol, poly(alkylenesulfides), phenolics, polyesters, asymmetric porous polymers, cross-linked olefin polymers, hydrophilic microporous homopolymers, copolymers or interpolymers having a reduced bulk density, and other similar materials, poly(urethane), cross-linked chain-extended poly(urethane), poly(imides), poly(benzimidazoles), collodion, regenerated proteins, semi-solid cross-linked poly(vinylpyrrolidone), monomeric, dimeric, oligomeric or long-chain, copolymers, block polymers, block co-polymers, polymers, PEG, dextran, modified dextran, polyvinylalcohol, polyvinylpyrollidone, polyacrylates, polymethacrylates, polyanhydrides, polypeptides, albumin, alginates, amino acids, thiols, amines and carboxylic acids or combinations thereof.

In various aspects, the present invention provides a microparticle coated microparticle cluster composition comprising: a microparticle cluster composition of the present invention and a coating of one or more second microparticles at least partially covering the microparticle cluster composition. Examples of suitable coating materials may include, but are not limited to, bovine serum albumin (BSA), lipids, polymers, and combinations thereof.

In various embodiments, the composition further comprises particles (e.g., microparticles) dispersed in the organic liquid. In some embodiments, the composition comprises an organic liquid comprising a plurality of particles (e.g., microparticles) of the present invention dispersed therein, and a coating material disposed around the exterior surface of the organic liquid. In one embodiment, the composition comprises an organic liquid and particles (e.g., microparticles) dispersed in organic liquid. In some embodiments, the composition further comprises a coating, which surrounds the exterior surface of organic liquid. Examples of organic liquids suitable for use in the microparticle cluster composition of the present disclosure may include, but are not limited to, perfluorocarbons, such as perfluorocarbons comprising about 5 to about 12 carbons, dodecafluoropentane (DDFP), commercially available from FluoroMed, L.P., Round Rock, Tex., and perfluororpentane.

The compositions of the present invention can be formulated in a pharmaceutically acceptable excipient, such as wetting agents, buffers, disintegrants, binders, fillers, flavoring agents and liquid carrier media such as sterile water, water/ethanol etc. The compositions of the present invention should be suitable for administration either by topical administration or oral administration or injection or inhalation or catheterization or instillation or transdermal introduction into any of the various body cavities including the alimentary canal, the vagina, the rectum, the bladder, the ureter, the urethra, the mouth, etc. For oral administration, the pH of the composition is preferably in the acid range (e.g., 2 to 7) and buffers or pH adjusting agents may be used. The contrast media may be formulated in conventional pharmaceutical administration forms, such as tablets, capsules, powders, solutions, dispersion, syrups, suppositories etc.

### Method of Preparation

The present invention also relates to methods, compositions, techniques, and strategies for making, purifying, characterizing, and using the polymers and particles (e.g., microparticles) described herein.

In one aspect, the invention provides a method of making a polymer of the invention. In one embodiment, the invention provides a method of making a polymer comprising the structure of Formula (I):

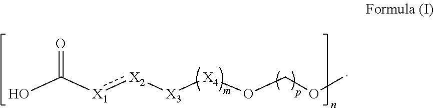

In some embodiments, each occurrence of X₁ is independently C═R₁, CR₂, or CR₃R₄. In some embodiments, each occurrence of X₂ is independently C═R₁, CR₂, or CR₃R₄. In some embodiments, each occurrence of X₃ is C═R₁ or CR₃R₄. In some embodiments, each occurrence of X₄ is independently C═R₁ or CR₃R₄.

In some embodiments, the bond between X₁ and X₂ is a single bond or a double bond. In some embodiments, when the bond between X₁ and X₂ is a single bond, X₁ and X₂ are each independently C═R₁ or CR₃R₄. In one embodiment, when the bond between X₁ and X₂ is a double bond, X₁ and X₂ are each C—R₂.

In some embodiments, R₁ is O, NH, or S. In one embodiment R₁ is O.

In some embodiments, R₂ is hydrogen, hydroxyl, carboxyl, alkyl, cycloalkyl, heterocycloalkyl, aryl, or heteroaryl. In one embodiment, R₂ is hydrogen. In one embodiment, R₂ is hydroxyl.

In some embodiments, R₃ is hydrogen, hydroxyl, carboxyl, alkyl, cycloalkyl, heterocycloalkyl, aryl, or heteroaryl. In one embodiment, R₃ is hydrogen. In one embodiment, R₃ is hydroxyl. In one embodiment, R₃ is carboxyl.

In some embodiments, R₄ is hydrogen, hydroxyl, carboxyl, alkyl, cycloalkyl, heterocycloalkyl, aryl, or heteroaryl. In one embodiment, R₄ is hydrogen. In one embodiment, R₄ is hydroxyl. In one embodiment, R₄ is carboxyl.

In some embodiments, m is an integer represented by 0, 1, 2, or 5.

In some embodiments, p is an integer from 1 to 50. In some embodiments, p is an integer from 1 to 15. In some embodiments, p is an integer from 1 to 10. For example, in one embodiment, p is an integer represented by 9. In another embodiment, p is an integer represented by 10.

In some embodiments, n is an integer from 1 to 1000.

In one embodiment, the method comprises reacting a compound or salt thereof comprising the structure of Formula (Ia) and a compound or salt thereof comprising the structure of Formula (Ib)

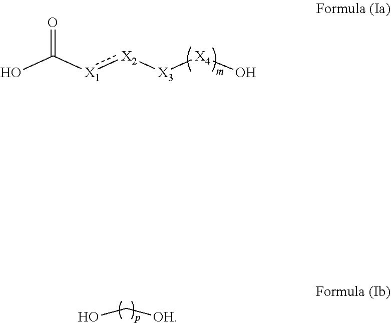

In some embodiments, each occurrence of X₁ is independently C═R₁, CR₂, or CR₃R₄. In some embodiments, each occurrence of X₂ is independently C═R₁, CR₂, or CR₃R₄. In some embodiments, each occurrence of X₃ is C═R₁ or CR₃R₄. In some embodiments, each occurrence of X₄ is independently C═R₁ or CR₃R₄.

In some embodiments, the bond between X₁ and X₂ is a single bond or a double bond. In some embodiments, when the bond between X₁ and X₂ is a single bond, X₁ and X₂ are each independently C═R₁ or CR₃R₄. In one embodiment, when the bond between X₁ and X₂ is a double bond, X₁ and X₂ are each C—R₂.

In some embodiments, R₁ is O, NH, or S. In one embodiment R₁ is O.

In some embodiments, R₂ is hydrogen, hydroxyl, carboxyl, alkyl, cycloalkyl, heterocycloalkyl, aryl, or heteroaryl. In one embodiment, R₂ is hydrogen. In one embodiment, R₂ is hydroxyl.

In some embodiments, R₃ is hydrogen, hydroxyl, carboxyl, alkyl, cycloalkyl, heterocycloalkyl, aryl, or heteroaryl. In one embodiment, R₃ is hydrogen. In one embodiment, R₃ is hydroxyl. In one embodiment, R₃ is carboxyl.

In some embodiments, R₄ is hydrogen, hydroxyl, carboxyl, alkyl, cycloalkyl, heterocycloalkyl, aryl, or heteroaryl. In one embodiment, R₄ is hydrogen. In one embodiment, R₄ is hydroxyl. In one embodiment, R₄ is carboxyl.

In some embodiments, m is an integer represented by 0, 1, 2, or 5.

In some embodiments, p is an integer from 1 to 50.

For example, in some embodiments, the compound having the structure of Formula (I) is αKG, succinic acid, citric acid, itaconic acid, sebacic acid, capric acid, glutaconic acid, heptadecanoic acid, hexanoic acid, decanoulcarnitine, or any combination thereof.

In one aspect, the invention provides a method of forming particles (e.g., microparticles) of the invention. In one embodiment, the method of forming the particle (e.g., microparticle) comprising the steps of: (a) mixing the compound comprising the structure of Formula (I) with an oil solvent and water solvent; and (b) forming the particle (e.g., microparticle) of the present invention in the water-oil emulsion.

In some embodiments, the preparing a water-oil emulsion by mixing the compound comprising the structure of Formula (I) with an oil solvent and water solvent further comprises at least one therapeutic agent, targeting moiety, or a combination thereof. In one aspect, the method further comprises (c) isolating the particle (e.g., microparticle).

### Methods of Treatment and Delivery of Therapeutic Agent

The compounds, particles (e.g., microparticles), and/or compositions thereof of the present invention can be used to deliver a therapeutic agent to a subject in need thereof. Thus, in one embodiment, the present invention provides a method of delivering a therapeutic agent to a subject in need thereof. In one embodiment, the method comprises administering at least one compound described herein to the subject. In one embodiment, the method comprises administering at least one particle (e.g., microparticle) described herein to the subject. In one embodiment, the method comprises administering at least one composition described herein to the subject.

In one embodiment, the method comprises administering a particle (e.g., microparticle) comprising a compound comprising the structure of Formula (I)-(XI) to a subject. In one embodiment, the particle (e.g., microparticle) releases the metabolite from the polymer. For example, in one embodiment, the method comprises administering a particle (e.g., microparticle) comprising a compound comprising the structure of Formula (II), wherein the particle (e.g., microparticle) releases αKG. In one embodiment, the method comprises administering a particle (e.g., microparticle) comprising a compound comprising the structure of Formula (III), wherein the particle (e.g., microparticle) releases citrate. In one embodiment, the method comprises administering a particle (e.g., microparticle) comprising a compound comprising the structure of Formula (IV), wherein the particle (e.g., microparticle) releases succinate. In one embodiment, the method comprises administering a particle (e.g., microparticle) comprising a compound comprising the structure of Formula (V), wherein the particle (e.g., microparticle) releases oxaloacetate. In one embodiment, the method comprises administering a particle (e.g., microparticle) comprising a compound comprising the structure of Formula (VI), wherein the particle (e.g., microparticle) releases isocitrate. In one embodiment, the method comprises administering a particle (e.g., microparticle) comprising a compound comprising the structure of Formula (VII), wherein the particle (e.g., microparticle) releases fumarate. In one embodiment, the method comprises administering a particle (e.g., microparticle) comprising a compound comprising the structure of Formula (VIII), wherein the particle (e.g., microparticle) releases malate. In one embodiment, the method comprises administering a particle (e.g., microparticle) comprising a compound comprising the structure of Formula (IX), wherein the particle (e.g., microparticle) releases citrate. In one embodiment, the method comprises administering a particle (e.g., microparticle) comprising a compound comprising the structure of Formula (X), wherein the particle (e.g., microparticle) releases isocitrate. In one embodiment, the method comprises administering a particle (e.g., microparticle) comprising a compound comprising the structure of Formula (XI), wherein the particle (e.g., microparticle) releases itaconate.

In one embodiment, the metabolite modulates the function of one or more immune cells. For example, in one embodiment, the metabolite activates dendritic cells (DCs). In one embodiment, the metabolite reduces pro-inflammatory cytokines and reactive oxygen species. In one embodiment, the metabolite induces alternate activation in macrophages.

In some embodiments, the particle (e.g., microparticle) delivers an additional therapeutic agent to the subject. For example, in one embodiment, the particle (e.g., microparticle) encapsulates an additional therapeutic agent and delivers the therapeutic agent to the subject. Thus, in one embodiment, the method of the invention delivers metabolite and an additional therapeutic agent to a subject in need thereof. In one embodiment, the therapeutic agent is any therapeutic agent described herein.

Any therapeutic agent or any combination of therapeutic agents disclosed herein may be administered to a subject to treat a disease or disorder. The therapeutic agents herein can be formulated in any number of ways, often according to various known formulations in the art or as disclosed or referenced herein.

In one embodiment, the invention provides a method for increasing the level of a metabolite in the subject. In one embodiment, the method comprises administering to the subject a particle (e.g., microparticle) of the invention. For example, in one embodiment, the method comprises administering a microparticle comprising a compound comprising the structure of Formula (I)-(XI), wherein the microparticle releases the metabolite from the polymer. Thus, in one embodiment, the method increases metabolites including, but not limited to, αKG, succinic acid, citric acid, spermidine, itaconic acid, sebacic acid, capric acid, glutaconic acid, heptadecanoic acid, hexanoic acid, decanoulcarnitine, or any combination thereof.

In some embodiments, the invention provides a method for increasing the level of interleukin 2 (IL-2), interleukin 10 (IL-10), interferon-gamma (IFN-γ), T cell to T helper (Th) cell ratio, T cell to CD4⁺ effector T cell ratio, regulatory T cell (Treg) to T helper type 1 (Th1) cell ratio, Th1 to T helper type 2 (Th2) cell ratio, T cell to CD8⁺ T cell ratio, Treg to CD8⁺ T cell ratio, Treg to type 1 CD8⁺ T (Tc1) cell ratio, and Treg to type 2 CD8⁺ T (Tc2) cell ratio, or any combination thereof. In some embodiments, the invention provides a method for decreasing the level of interleukin 12 (IL-12) and IL-12 p70 subunit gene in the subject, or any combination thereof. In one embodiment, the method comprises administering to the subject a particle (e.g., microparticle) of the invention. For example, in one embodiment, the method comprises administering a microparticle comprising a compound comprising the structure of Formula (I)-(XI).

In some embodiments, the invention provides a method for decreasing the level of a pro-inflammatory cytokine; increasing the level of an anti-inflammatory cytokine; increasing the level of a T regulatory cell; or any combination thereof. In some embodiments, the method decreases the level of a pro-inflammatory cytokine; increases the level of an anti-inflammatory cytokine; increases the level of a T regulatory cell; or any combination thereof. In some embodiments, the method decreases the level of a pro-inflammatory cytokine; increases the level of an anti-inflammatory cytokine; increases the level of a T regulatory cell; or any combination thereof.

In one aspect the present invention provides a method of treating or preventing a disease or disorder associated with increased level of a pro-inflammatory cytokine; decreased level of an anti-inflammatory cytokine; decreased level of a T regulatory cell; or any combination thereof in a subject in need thereof, the method comprising administering at least one compound, particle (e.g., microparticle), or composition described herein to the subject.

In various aspects, the present invention provides a method of treating a disease or disorder associated with increased level of a pro-inflammatory cytokine; decreased level of an anti-inflammatory cytokine; decreased level of a T regulatory cell; or any combination thereof in a subject in need thereof comprising the steps of: administering one or more compounds, particles (e.g., microparticles), or compositions described herein to the subject; phagocyting the compounds, particles (e.g., microparticles), or compositions by a cell; releasing of a therapeutic agent into the cell from the compounds, particles (e.g., microparticles), or compositions; and facilitating decrease in the level of a pro-inflammatory cytokine; increase in the level of an anti-inflammatory cytokine; increase in the level of a T regulatory cell; or any combination thereof.

In certain embodiments, the method of treating a disease or disorder comprises a “triggered” functionality. In other words, the system may remain inert in the body until specifically triggered. In some embodiments, the compound or particle (e.g., microparticle) is used advantageously in therapeutic applications such as to first target the compound or particle (e.g., microparticle) to a specified location, and then trigger them into an activated state. Sometimes referred to as a “dual targeted delivery system,” this feature may minimize the side effects of systemic therapeutic agents. For example, in some embodiments, upon delivering the compound or particle (e.g., microparticle) to a specific cell, a reagent, such as water, proton, acid, or protonated water, may be applied to the cell thereby causing the release of a therapeutic agent from the compound or particle (e.g., microparticle). In some embodiments, this may provide a clinician the ability to control and visualize drug therapy noninvasively.

In some embodiments, the size (e.g., average diameter of the particle, such as microparticle) of the compound, particle (e.g., microparticle), or composition of the present invention allows for passive diffusion into cells. In some embodiments, where the compound, particle (e.g., microparticle), or composition is on a smaller scale, the small size (e.g., average diameter of the particle, such as microparticle) allows the compound, particle (e.g., microparticle), or compositions to travel almost anywhere in the body where therapy may need to be performed. For example, in some embodiments, the method comprises compounds or microparticles that act as a hydrolysis triggered therapeutic agent delivery and therapeutic agent release systems.

In some embodiments, the compound, particle (e.g., microparticle), or composition undergo uptake into biological sample. In some embodiments, the compound, particle (e.g., microparticle), or composition undergo uptake into macrophage cells. In some embodiments, the compound, particle (e.g., microparticle), or composition undergo uptake into dendritic cells. For example, in some embodiments, the compound, microparticle, or composition can be coated with dextran to target the macrophage cells, since macrophages have dextran receptors. In various embodiments, the method further comprises allowing the compound, microparticle, or composition to accumulate in a region of the biological tissue, wherein the targeting domain facilitated accumulation of the compound, microparticle, or composition in the region.

In one aspect, the present invention provides a method of enhancing biological tissue growth in a subject in need thereof, the method comprising administering at least one compound, particle (e.g., microparticle), or composition described herein to the subject.

In another aspect, the present invention provides a method of regenerating a biological tissue in a subject in need thereof, the method comprising administering at least one compound, particle (e.g., microparticle), or composition described herein to the subject.

Examples of such biological tissues include but a connective tissue, epithelial tissue, muscular tissue, and nervous tissue.

In another aspect, the present invention provides a method of enhancing biological wound healing or wound closure in a subject in need thereof, the method comprising administering at least one compound, particle (e.g., microparticle), or composition described herein to the subject.

In another aspect, the present invention provides a method of facilitating wound healing in a subject in need thereof, the method comprising administering at least one compound, particle (e.g., microparticle), or composition described herein to the subject.

In various aspects, the compound, particle (e.g., microparticle), or composition of the present invention can be used alone or in combination with a therapeutic agent to deliver a therapeutic agent payload to a target cell. Often, the therapeutic agent may be released based on the degradation of, e.g., a controlled release biodegradable matrix and/or polymer. However, it has been found that the compounds or particles (e.g., microparticles) of the present invention can also deliver their payload by hydrolysis disruption of the compounds or particles (e.g., microparticles).

In one embodiment, the invention provides a method of treating a disease or disorder in a subject in need thereof. In one embodiment, the method comprises administering a compound, particle (e.g., microparticle), or composition to the subject. In one embodiment, the disease or disorder is a disease or disorder associated with abnormal immune cell function.

The preferred dosage of the compound or particle (e.g., microparticle) will vary according to a number of factors, such as the administration route, the age, weight and species of the subject, but in general containing in the order of from 1 μmol/kg to 1 mmol/kg bodyweight of the compound or particle (e.g., microparticle).

Administration may be topical, parenteral (e.g., intravenously, intraperitoneally, intraarterially, intramuscularly, interstitially, subcutaneously, transdermally, or intrasternally), or into an externally voiding body cavity (e.g., the gastrointestinal tract, rectum, bladder, uterus, vagina, nose, ears or lungs), peritoneally, orally, intradermal, ocular, in an animate human or non-human (e.g., mammalian, reptilian or avian) body.

### Pharmaceutical Compositions

The compounds, particles (e.g., microparticles), or compositions of the invention can be formulated and administered to a subject, as now described. The invention encompasses the preparation and use of pharmaceutical compositions comprising the compound, particle (e.g., microparticle), and/or compositions of the invention useful for the delivery of a therapeutic agent, such as metabolite, to a cell (e.g., delivery of αKG to a dendritic cell). The invention also encompasses the preparation and use of pharmaceutical compositions comprising the compound, particle (e.g., microparticle), and/or compositions of the invention useful for the treatment of a disease or disorder (e.g., any disease or disorder associated with increased level of a pro-inflammatory cytokine; decreased level of an anti-inflammatory cytokine; decreased level of a T regulatory cell; or any combination thereof). The invention also encompasses the preparation and use of pharmaceutical compositions comprising the compound, microparticle, and/or compositions of the invention useful for the growth or regeneration of biological tissue (e.g., wound healing).

Such a pharmaceutical composition may consist of the active ingredient alone, in a form suitable for administration to a subject, or the pharmaceutical composition may comprise the active ingredient and one or more pharmaceutically acceptable carriers, one or more additional ingredients, or some combination of these. The active ingredient may be present in the pharmaceutical composition in the form of a physiologically acceptable ester or salt, such as in combination with a physiologically acceptable cation or anion, as is well known in the art.

The pharmaceutical compositions useful for practicing the invention may be administered to deliver a dose of between about 0.01 ng/kg/day and 500 mg/kg/day.

In various embodiments, the pharmaceutical compositions useful in the methods of the invention may be administered, by way of example, systemically, parenterally, or topically, such as, in oral formulations, inhaled formulations, including solid or aerosol, and by topical or other similar formulations. In addition to the appropriate therapeutic composition, such pharmaceutical compositions may contain pharmaceutically acceptable carriers and other ingredients known to enhance and facilitate drug administration. Other possible formulations, such as nanoparticles, liposomes, resealed erythrocytes, and immunologically based systems may also be used to administer an appropriate modulator thereof, according to the methods of the invention.

As used herein, the term “physiologically acceptable” ester or salt means an ester or salt form of the active ingredient which is compatible with any other ingredients of the pharmaceutical composition, which is not deleterious to the subject to which the composition is to be administered.

The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

Although the descriptions of pharmaceutical compositions provided herein are principally directed to pharmaceutical compositions which are suitable for ethical administration to humans, it will be understood by the skilled artisan that such compositions are generally suitable for administration to animals, patients, and subjects of all sorts. Modification of pharmaceutical compositions suitable for administration to humans in order to render the compositions suitable for administration to various animals and patients is well understood, and the ordinarily skilled veterinary pharmacologist can design and perform such modification with merely ordinary, if any, experimentation.

Pharmaceutical compositions that are useful in the methods of the invention may be prepared, packaged, or sold in formulations suitable for oral, rectal, vaginal, parenteral, topical, pulmonary, intranasal, buccal, intravenous, ophthalmic, intrathecal and other known routes of administration. Other contemplated formulations include projected nanoparticles, liposomal preparations, resealed erythrocytes containing the active ingredient, and immunologically-based formulations.

A pharmaceutical composition of the invention may be prepared, packaged, or sold in bulk, as a single unit dose, or as a plurality of single unit doses. As used herein, a “unit dose” is discrete amount of the pharmaceutical composition comprising a predetermined amount of the active ingredient. The amount of the active ingredient is generally equal to the dosage of the active ingredient which would be administered to a subject or a convenient fraction of such a dosage such as, for example, one-half or one-third of such a dosage.

The relative amounts of the active ingredient, the pharmaceutically acceptable carrier, and any additional ingredients in a pharmaceutical composition of the invention will vary, depending upon the identity, size, and condition of the subject treated and further depending upon the route by which the composition is to be administered. By way of example, the composition may comprise between 0.1% and 100% (w/w) active ingredient.

In addition to the active ingredient, a pharmaceutical composition of the invention may further comprise one or more additional pharmaceutically active agents.

Controlled- or sustained-release formulations of a pharmaceutical composition of the invention may be made using conventional technology.

A formulation of a pharmaceutical composition of the invention suitable for oral administration may be prepared, packaged, or sold in the form of a discrete solid dose unit including, but not limited to, a tablet, a hard or soft capsule, a cachet, a troche, or a lozenge, each containing a predetermined amount of the active ingredient. Other formulations suitable for oral administration include, but are not limited to, a powdered or granular formulation, an aqueous or oily suspension, an aqueous or oily solution, or an emulsion.

A tablet comprising the active ingredient may, for example, be made by compressing or molding the active ingredient, optionally with one or more additional ingredients. Compressed tablets may be prepared by compressing, in a suitable device, the active ingredient in a free-flowing form such as a powder or granular preparation, optionally mixed with one or more of a binder, a lubricant, an excipient, a surface active agent, and a dispersing agent. Molded tablets may be made by molding, in a suitable device, a mixture of the active ingredient, a pharmaceutically acceptable carrier, and at least sufficient liquid to moisten the mixture. Pharmaceutically acceptable excipients used in the manufacture of tablets include, but are not limited to, inert diluents, granulating and disintegrating agents, binding agents, and lubricating agents. Known dispersing agents include, but are not limited to, potato starch and sodium starch glycolate. Known surface active agents include, but are not limited to, sodium lauryl sulphate. Known diluents include, but are not limited to, calcium carbonate, sodium carbonate, lactose, microcrystalline cellulose, calcium phosphate, calcium hydrogen phosphate, and sodium phosphate. Known granulating and disintegrating agents include, but are not limited to, corn starch and alginic acid. Known binding agents include, but are not limited to, gelatin, acacia, pre-gelatinized maize starch, polyvinylpyrrolidone, and hydroxypropyl methylcellulose. Known lubricating agents include, but are not limited to, magnesium stearate, stearic acid, silica, and talc.

Tablets may be non-coated or they may be coated using known methods to achieve delayed disintegration in the gastrointestinal tract of a subject, thereby providing sustained release and absorption of the active ingredient. By way of example, a material such as glyceryl monostearate or glyceryl distearate may be used to coat tablets. Further by way of example, tablets may be coated using methods described in U.S. Pat. Nos. 4,256,108; 4,160,452; and U.S. Pat. No. 4,265,874 to form osmotically-controlled release tablets. Tablets may further comprise a sweetening agent, a flavoring agent, a coloring agent, a preservative, or some combination of these in order to provide pharmaceutically elegant and palatable preparation.

Hard capsules comprising the active ingredient may be made using a physiologically degradable composition, such as gelatin. Such hard capsules comprise the active ingredient, and may further comprise additional ingredients including, for example, an inert solid diluent such as calcium carbonate, calcium phosphate, or kaolin.

Soft gelatin capsules comprising the active ingredient may be made using a physiologically degradable composition, such as gelatin. Such soft capsules comprise the active ingredient, which may be mixed with water or an oil medium such as peanut oil, liquid paraffin, or olive oil.

Liquid formulations of a pharmaceutical composition of the invention which are suitable for oral administration may be prepared, packaged, and sold either in liquid form or in the form of a dry product intended for reconstitution with water or another suitable vehicle prior to use.

Liquid suspensions may be prepared using conventional methods to achieve suspension of the active ingredient in an aqueous or oily vehicle. Aqueous vehicles include, for example, water and isotonic saline. Oily vehicles include, for example, almond oil, oily esters, ethyl alcohol, vegetable oils such as arachis, olive, sesame, or coconut oil, fractionated vegetable oils, and mineral oils such as liquid paraffin. Liquid suspensions may further comprise one or more additional ingredients including, but not limited to, suspending agents, dispersing or wetting agents, emulsifying agents, demulcents, preservatives, buffers, salts, flavorings, coloring agents, and sweetening agents. Oily suspensions may further comprise a thickening agent.

Known suspending agents include, but are not limited to, sorbitol syrup, hydrogenated edible fats, sodium alginate, polyvinylpyrrolidone, gum tragacanth, gum acacia, and cellulose derivatives such as sodium carboxymethylcellulose, methylcellulose, and hydroxypropylmethylcellulose. Known dispersing or wetting agents include, but are not limited to, naturally-occurring phosphatides such as lecithin, condensation products of an alkylene oxide with a fatty acid, with a long chain aliphatic alcohol, with a partial ester derived from a fatty acid and a hexitol, or with a partial ester derived from a fatty acid and a hexitol anhydride (e.g. polyoxyethylene stearate, heptadecaethyleneoxycetanol, polyoxyethylene sorbitol monooleate, and polyoxyethylene sorbitan monooleate, respectively). Known emulsifying agents include, but are not limited to, lecithin and acacia. Known preservatives include, but are not limited to, methyl, ethyl, or n-propyl-para-hydroxybenzoates, ascorbic acid, and sorbic acid. Known sweetening agents include, for example, glycerol, propylene glycol, sorbitol, sucrose, and saccharin. Known thickening agents for oily suspensions include, for example, beeswax, hard paraffin, and cetyl alcohol.

Liquid solutions of the active ingredient in aqueous or oily solvents may be prepared in substantially the same manner as liquid suspensions, the primary difference being that the active ingredient is dissolved, rather than suspended in the solvent. Liquid solutions of the pharmaceutical composition of the invention may comprise each of the components described with regard to liquid suspensions, it being understood that suspending agents will not necessarily aid dissolution of the active ingredient in the solvent. Aqueous solvents include, for example, water and isotonic saline. Oily solvents include, for example, almond oil, oily esters, ethyl alcohol, vegetable oils such as arachis, olive, sesame, or coconut oil, fractionated vegetable oils, and mineral oils such as liquid paraffin.

Powdered and granular formulations of a pharmaceutical preparation of the invention may be prepared using known methods. Such formulations may be administered directly to a subject, used, for example, to form tablets, to fill capsules, or to prepare an aqueous or oily suspension or solution by addition of an aqueous or oily vehicle thereto. Each of these formulations may further comprise one or more of dispersing or wetting agent, a suspending agent, and a preservative. Additional excipients, such as fillers and sweetening, flavoring, or coloring agents, may also be included in these formulations.

A pharmaceutical composition of the invention may also be prepared, packaged, or sold in the form of oil-in-water emulsion or a water-in-oil emulsion. The oily phase may be a vegetable oil such as olive or arachis oil, a mineral oil such as liquid paraffin, or a combination of these. Such compositions may further comprise one or more emulsifying agents such as naturally occurring gums such as gum acacia or gum tragacanth, naturally-occurring phosphatides such as soybean or lecithin phosphatide, esters or partial esters derived from combinations of fatty acids and hexitol anhydrides such as sorbitan monooleate, and condensation products of such partial esters with ethylene oxide such as polyoxyethylene sorbitan monooleate. These emulsions may also contain additional ingredients including, for example, sweetening or flavoring agents.

Methods for impregnating or coating a material with a chemical composition are known in the art, and include, but are not limited to methods of depositing or binding a chemical composition onto a surface, methods of incorporating a chemical composition into the structure of a material during the synthesis of the material (i.e. such as with a physiologically degradable material), and methods of absorbing an aqueous or oily solution or suspension into an absorbent material, with or without subsequent drying.

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, cutaneous, subcutaneous, intraperitoneal, intravenous, intramuscular, intracisternal injection, and kidney dialytic infusion techniques.

Formulations of a pharmaceutical composition suitable for parenteral administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multi-dose containers containing a preservative. Formulations for parenteral administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents. In one embodiment of a formulation for parenteral administration, the active ingredient is provided in dry (i.e. powder or granular) form for reconstitution with a suitable vehicle (e.g., sterile pyrogen-free water) prior to parenteral administration of the reconstituted composition.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally-acceptable diluent or solvent, such as water or 1,3-butane diol, for example. Other acceptable diluents and solvents include, but are not limited to, Ringer's solution, isotonic sodium chloride solution, and fixed oils such as synthetic mono- or di-glycerides. Other parentally-administrable formulations which are useful include those which comprise the active ingredient in microcrystalline form, in a liposomal preparation, or as a component of a biodegradable polymer systems. Compositions for sustained release or implantation may comprise pharmaceutically acceptable polymeric or hydrophobic materials such as an emulsion, an ion exchange resin, a sparingly soluble polymer, or a sparingly soluble salt.

Formulations suitable for topical administration include, but are not limited to, liquid or semi-liquid preparations such as liniments, lotions, oil-in-water or water-in-oil emulsions such as creams, ointments or pastes, and solutions or suspensions. Topically-administrable formulations may, for example, comprise from about 1% to about 10% (w/w) active ingredient, although the concentration of the active ingredient may be as high as the solubility limit of the active ingredient in the solvent. Formulations for topical administration may further comprise one or more of the additional ingredients described herein.

A pharmaceutical composition of the invention may be prepared, packaged, or sold in a formulation suitable for pulmonary administration via the buccal cavity. Such a formulation may comprise dry particles which comprise the active ingredient and which have a diameter in the range from about 0.5 to about 7 nanometers, and preferably from about 1 to about 6 nanometers. Such compositions are conveniently in the form of dry powders for administration using a device comprising a dry powder reservoir to which a stream of propellant may be directed to disperse the powder or using a self-propelling solvent/powder-dispensing container such as a device comprising the active ingredient dissolved or suspended in a low-boiling propellant in a sealed container. Preferably, such powders comprise particles wherein at least 98% of the particles by weight have a diameter greater than 0.5 nanometers and at least 95% of the particles by number have a diameter less than 7 nanometers. More preferably, at least 95% of the particles by weight have a diameter greater than 1 nanometer and at least 90% of the particles by number have a diameter less than 6 nanometers. Dry powder compositions preferably include a solid fine powder diluent such as sugar and are conveniently provided in a unit dose form.

Low boiling propellants generally include liquid propellants having a boiling point of below 65° F. at atmospheric pressure. Generally the propellant may constitute 50 to 99.9% (w/w) of the composition, and the active ingredient may constitute 0.1 to 20% (w/w) of the composition. The propellant may further comprise additional ingredients such as a liquid non-ionic or solid anionic surfactant or a solid diluent (preferably having a particle size of the same order as particles comprising the active ingredient).

Pharmaceutical compositions of the invention formulated for pulmonary delivery may also provide the active ingredient in the form of droplets of a solution or suspension. Such formulations may be prepared, packaged, or sold as aqueous or dilute alcoholic solutions or suspensions, optionally sterile, comprising the active ingredient, and may conveniently be administered using any nebulization or atomization device. Such formulations may further comprise one or more additional ingredients including, but not limited to, a flavoring agent such as saccharin sodium, a volatile oil, a buffering agent, a surface active agent, or a preservative such as methylhydroxybenzoate. The droplets provided by this route of administration preferably have an average diameter in the range from about 0.1 to about 200 nanometers.

The formulations described herein as being useful for pulmonary delivery are also useful for intranasal delivery of a pharmaceutical composition of the invention.

Another formulation suitable for intranasal administration is a coarse powder comprising the active ingredient and having an average particle from about 0.2 to 500 micrometers.

Such a formulation is administered in the manner in which snuff is taken i.e. by rapid inhalation through the nasal passage from a container of the powder held close to the nares. Formulations suitable for nasal administration may, for example, comprise from about as little as 0.1% (w/w) and as much as 100% (w/w) of the active ingredient, and may further comprise one or more of the additional ingredients described herein.

A pharmaceutical composition of the invention may be prepared, packaged, or sold in a formulation suitable for buccal administration. Such formulations may, for example, be in the form of tablets or lozenges made using conventional methods, and may, for example, contain 0.1 to 20% (w/w) active ingredient, the balance comprising an orally dissolvable or degradable composition and, optionally, one or more of the additional ingredients described herein. Alternately, formulations suitable for buccal administration may comprise a powder or an aerosolized or atomized solution or suspension comprising the active ingredient. Such powdered, aerosolized, or aerosolized formulations, when dispersed, preferably have an average particle or droplet size in the range from about 0.1 nanometers to about 2000 micrometers, and may further comprise one or more of the additional ingredients described herein.

A pharmaceutical composition of the invention may be prepared, packaged, or sold in a formulation suitable for ophthalmic administration. Such formulations may, for example, be in the form of eye drops including, for example, a 0.1-1.0% (w/w) solution or suspension of the active ingredient in an aqueous or oily liquid carrier. Such drops may further comprise buffering agents, salts, or one or more other of the additional ingredients described herein. Other ophthalmically-administrable formulations which are useful include those which comprise the active ingredient in microcrystalline form or in a liposomal preparation.

As used herein, “additional ingredients” include, but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additional ingredients” which may be included in the pharmaceutical compositions of the invention are known in the art and described, for example in Genaro, ed., 1985, Remington's Pharmaceutical Sciences, Mack Publishing Co., Easton, Pa.

Typically dosages of the compound of the invention which may be administered to an animal or patient, preferably a human, range in amount from about 0.01 mg to about 100 g per kilogram of body weight of the animal or patient. While the precise dosage administered will vary depending upon any number of factors, including, but not limited to, the type of animal and type of disease state being treated, the age of the animal or patient and the route of administration. Preferably, the dosage of the compound will vary from about 0.01 mg to about 500 mg per kilogram of body weight of the animal or patient. The compound can be administered to an animal or patient as frequently as several times daily, or it can be administered less frequently, such as once a day, once a week, once every two weeks, once a month, or even less frequently, such as once every several months or even once a year or less. The frequency of the dose will be readily apparent to the skilled artisan and will depend upon any number of factors, such as, but not limited to, the type and severity of the disease being treated, the type and age of the animal, patient, etc.

Administration of the compounds of the present invention or the compositions thereof may be continuous or intermittent, depending, for example, upon the recipient's physiological condition, whether the purpose of the administration is therapeutic or prophylactic, and other factors known to skilled practitioners. The administration of the agents of the invention may be essentially continuous over a preselected period of time or may be in a series of spaced doses. Both local and systemic administration is contemplated. The amount administered will vary depending on various factors including, but not limited to, the composition chosen, the particular disease, the weight, the physical condition, and the age of the mammal, and whether prevention or treatment is to be achieved. Such factors can be readily determined by the clinician employing animal models or other test systems which are well known to the art.

One or more suitable unit dosage forms having the therapeutic agent(s) of the invention, which, as discussed below, may optionally be formulated for sustained release, can be administered by a variety of routes including parenteral, including by intravenous and intramuscular routes, as well as by direct injection into the diseased tissue. For example, the therapeutic agent may be directly injected into the muscle. The formulations may, where appropriate, be conveniently presented in discrete unit dosage forms and may be prepared by any of the methods well known to pharmacy. Such methods may include the step of bringing into association the therapeutic agent with liquid carriers, solid matrices, semi-solid carriers, finely divided solid carriers or combinations thereof, and then, if necessary, introducing or shaping the product into the desired delivery system.

When the therapeutic agents of the invention are prepared for administration, they are preferably combined with a pharmaceutically acceptable carrier, diluent or excipient to form a pharmaceutical formulation, or unit dosage form. The total active ingredients in such formulations include from 0.1 to 99.9% by weight of the formulation. A “pharmaceutically acceptable” is a carrier, diluent, excipient, and/or salt that is compatible with the other ingredients of the formulation, and not deleterious to the recipient thereof. The active ingredient for administration may be present as a powder or as granules; as a solution, a suspension or an emulsion.

Pharmaceutical formulations containing the therapeutic agents of the invention can be prepared by procedures known in the art using well known and readily available ingredients. The therapeutic agents of the invention can also be formulated as solutions appropriate for parenteral administration, for instance by intramuscular, subcutaneous or intravenous routes.

The pharmaceutical formulations of the therapeutic agents of the invention can also take the form of an aqueous or anhydrous solution or dispersion, or alternatively the form of an emulsion or suspension.

Thus, the therapeutic agent may be formulated for parenteral administration (e.g., by injection, for example, bolus injection or continuous infusion) and may be presented in unit dose form in ampules, pre-filled syringes, small volume infusion containers or in multi-dose containers with an added preservative. The active ingredients may take such forms as suspensions, solutions, or emulsions in oily or aqueous vehicles, and may contain formulatory agents such as suspending, stabilizing and/or dispersing agents. Alternatively, the active ingredients may be in powder form, obtained by aseptic isolation of sterile solid or by lyophilization from solution, for constitution with a suitable vehicle, e.g., sterile, pyrogen-free water, before use.

It will be appreciated that the unit content of active ingredient or ingredients contained in an individual aerosol dose of each dosage form need not in itself constitute an effective amount for treating the particular indication or disease since the necessary effective amount can be reached by administration of a plurality of dosage units. Moreover, the effective amount may be achieved using less than the dose in the dosage form, either individually, or in a series of administrations.

The pharmaceutical formulations of the present invention may include, as optional ingredients, pharmaceutically acceptable carriers, diluents, solubilizing or emulsifying agents, and salts of the type that are well-known in the art. Specific non-limiting examples of the carriers and/or diluents that are useful in the pharmaceutical formulations of the present invention include water and physiologically acceptable buffered saline solutions, such as phosphate buffered saline solutions pH 7.0-8.0.

In general, water, suitable oil, saline, aqueous dextrose (glucose), and related sugar solutions and glycols such as propylene glycol or polyethylene glycols are suitable carriers for parenteral solutions. Solutions for parenteral administration contain the active ingredient, suitable stabilizing agents and, if necessary, buffer substances. Antioxidizing agents such as sodium bisulfate, sodium sulfite or ascorbic acid, either alone or combined, are suitable stabilizing agents. Also used are citric acid and its salts and sodium Ethylenediaminetetraacetic acid (EDTA). In addition, parenteral solutions can contain preservatives such as benzalkonium chloride, methyl- or propyl-paraben and chlorobutanol. Suitable pharmaceutical carriers are described in Remington's Pharmaceutical Sciences, a standard reference text in this field.

The active ingredients of the invention may be formulated to be suspended in a pharmaceutically acceptable composition suitable for use in mammals and in particular, in humans. Such formulations include the use of adjuvants such as muramyl dipeptide derivatives (MDP) or analogs that are described in U.S. Pat. Nos. 4,082,735; 4,082,736; 4,101,536; 4,185,089; 4,235,771; and 4,406,890. Other adjuvants, which are useful, include alum (Pierce Chemical Co.), lipid A, trehalose dimycolate and dimethyldioctadecylammonium bromide (DDA), Freund's adjuvant, and IL-12. Other components may include a polyoxypropylene-polyoxyethylene block polymer (Pluronic®), a non-ionic surfactant, and a metabolizable oil such as squalene (U.S. Pat. No. 4,606,918).

Additionally, standard pharmaceutical methods can be employed to control the duration of action. These are well known in the art and include control release preparations and can include appropriate macromolecules, for example polymers, polyesters, polyamino acids, polyvinyl, pyrolidone, ethylenevinylacetate, methyl cellulose, carboxymethyl cellulose or protamine sulfate. The concentration of macromolecules as well as the methods of incorporation can be adjusted in order to control release. Additionally, the agent can be incorporated into particles of polymeric materials such as polyesters, polyamino acids, hydrogels, poly(lactic acid) or ethylenevinylacetate copolymers. In addition to being incorporated, these agents can also be used to trap the compound in microcapsules.

Accordingly, the composition of the present invention may be delivered via various routes and to various sites in a mammal body to achieve a particular effect (see, e.g., Rosenfeld et al., 1991; Rosenfeld et al., 1991a; Jaffe et al., supra; Berkner, supra). One skilled in the art will recognize that although more than one route can be used for administration, a particular route can provide a more immediate and more effective reaction than another route. In one embodiment, the composition described above is administered to the subject by subretinal injection. In other embodiments, the composition is administered by intravitreal injection. Other forms of administration that may be useful in the methods described herein include, but are not limited to, direct delivery to a desired organ (e.g., the eye), oral, inhalation, intranasal, intraperitoneal, intratracheal, intravenous, intramuscular, topical, subcutaneous, intradermal, and other parental routes of administration. Additionally, routes of administration may be combined, if desired. In another embodiments, route of administration is subretinal injection or intravitreal injection.

The active ingredients of the present invention can be provided in unit dosage form wherein each dosage unit, e.g., a teaspoonful, tablet, solution, or suppository, contains a predetermined amount of the composition, alone or in appropriate combination with other active agents. The term “unit dosage form” as used herein refers to physically discrete units suitable as unitary dosages for human and mammal subjects, each unit containing a predetermined quantity of the compositions of the present invention, alone or in combination with other active agents, calculated in an amount sufficient to produce the desired effect, in association with a pharmaceutically acceptable diluent, carrier, or vehicle, where appropriate. The specifications for the unit dosage forms of the present invention depend on the particular effect to be achieved and the particular pharmacodynamics associated with the composition in the particular host.

These methods described herein are by no means all-inclusive, and further methods to suit the specific application will be apparent to the ordinary skilled artisan. Moreover, the effective amount of the compositions can be further approximated through analogy to compounds known to exert the desired effect.

It will be understood by those of skill in the art that numerous and various modifications can be made without departing from the spirit of the present disclosure. Therefore, it should be clearly understood that the forms disclosed herein are illustrative only and are not intended to limit the scope of the present disclosure.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the present invention and practice the claimed methods. The following working examples therefore, specifically point out the preferred embodiments of the present invention, and are not to be construed as limiting in any way the remainder of the disclosure.

### Example 1: Poly-α-Ketoglutarate (pαKG or paKG) Polymers Generated from α-Ketoglutarate (αKG) and Capable of Releasing αKG

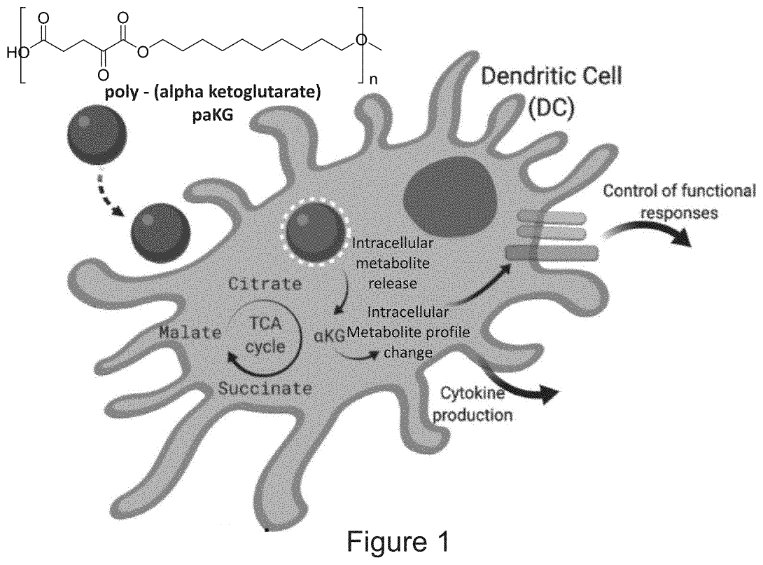

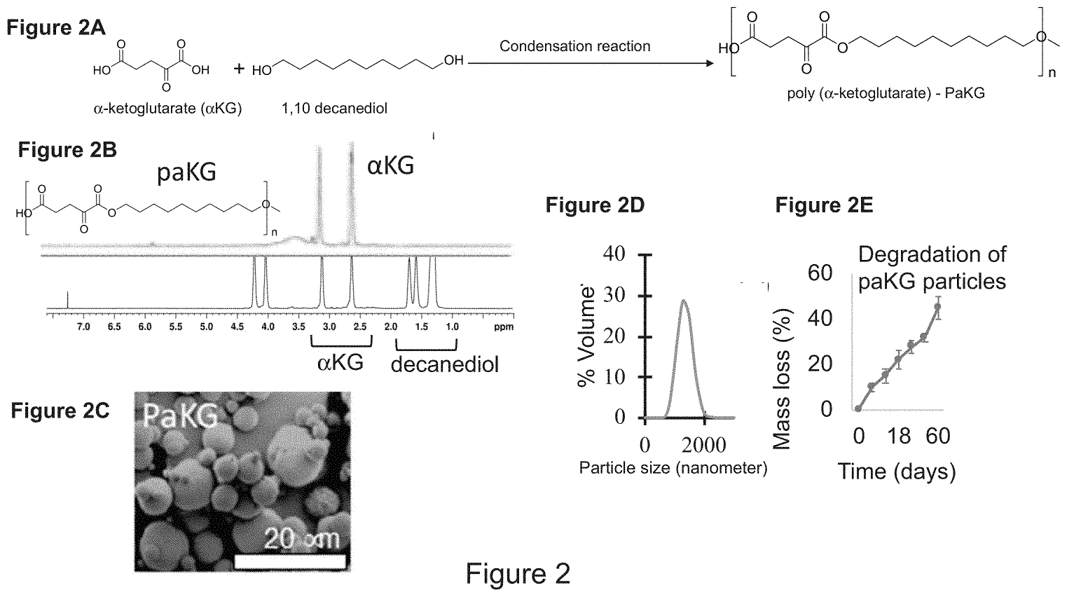

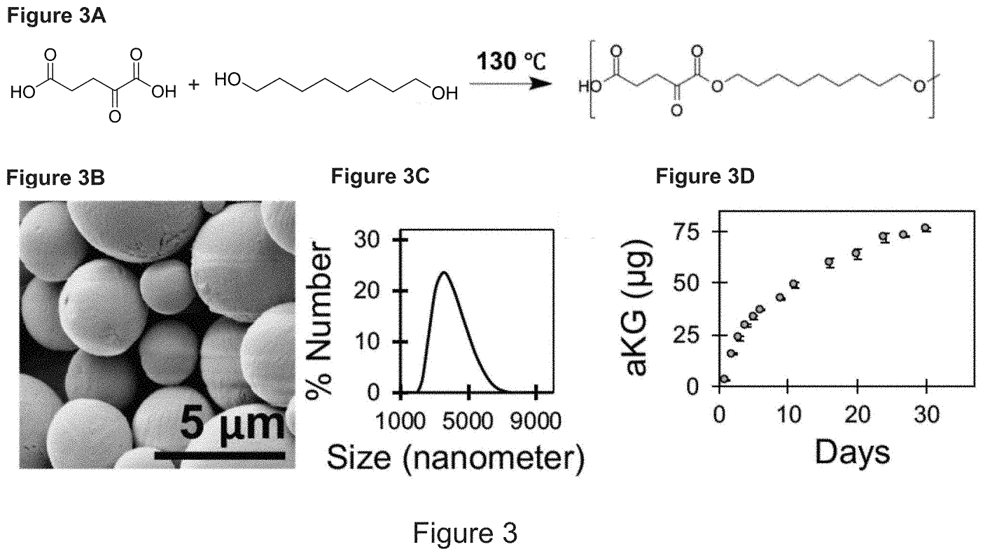

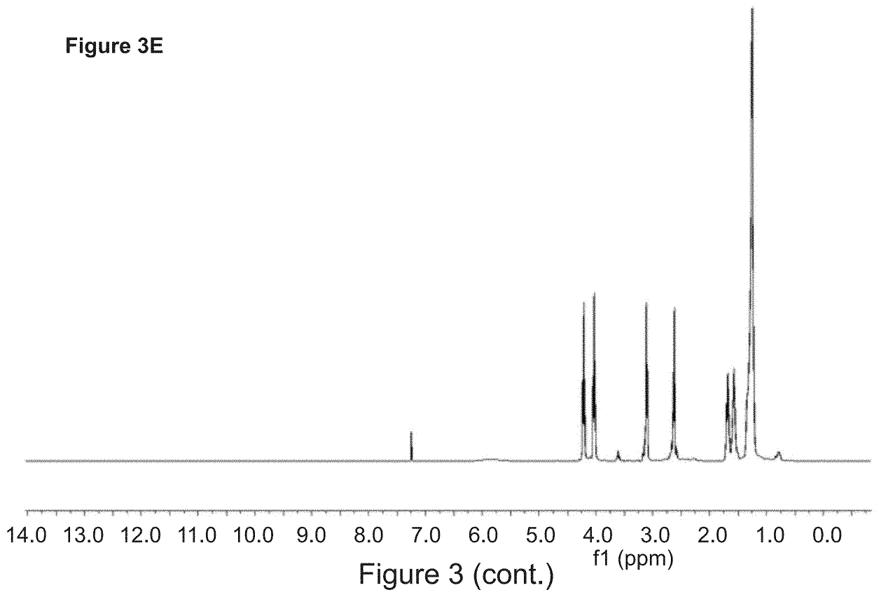

In order to develop a drug delivery vehicle that does not activate the dendritic cells (DCs) and are capable of delivering both antigens and immunosuppressive agents, pαKG polymers were synthesized from the monomers αKG and 1,10-decanediol (FIG. 1). αKG is known to favor M2 (suppressive) phenotype rather than M1 (activated) phenotype in macrophages. However, delivery of αKG to modulate the metabolism of immune cells is non-trivial, as this molecule gets metabolized quickly, and diffuses away from the injection site, and thus needs to be provided via multiple injection. Condensation reaction between αKG and 1,10-decanethiol was utilized to generate pαKG polymers, which were degradable via hydrolysis and provided release of αKG (FIG. 2A, FIG. 2B, and FIG. 3A). Moreover, microparticles were generated out of these polymers using a water-oil emulsion with dichloromethane as the oil phase and water as the aqueous phase. The average size of these particles was determined to be 1 μm using scanning electron microscopy and dynamic light scattering (FIG. 2C, FIG. 2D, FIG. 3B, and FIG. 3C). Moreover, the particles degraded slowly in phosphate buffered saline solution over 60 days losing approximately 50% of their mass (FIG. 2E and FIG. 3D) and released αKG in a sustained manner (FIG. 2B—representative green ¹H NMR—day 10 release shown). These data indicated that pαKG were generated, which can release αKG from microparticles.

### Example 2: pαKG Particles were Phagocytosed by DCs and Did not Activate them In Vitro

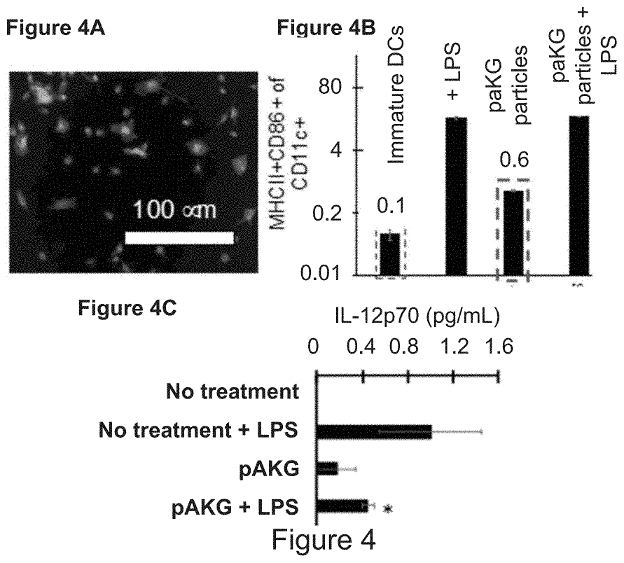

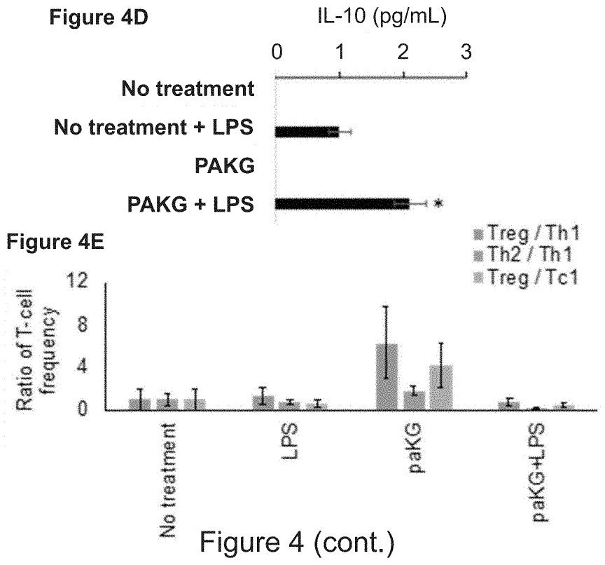

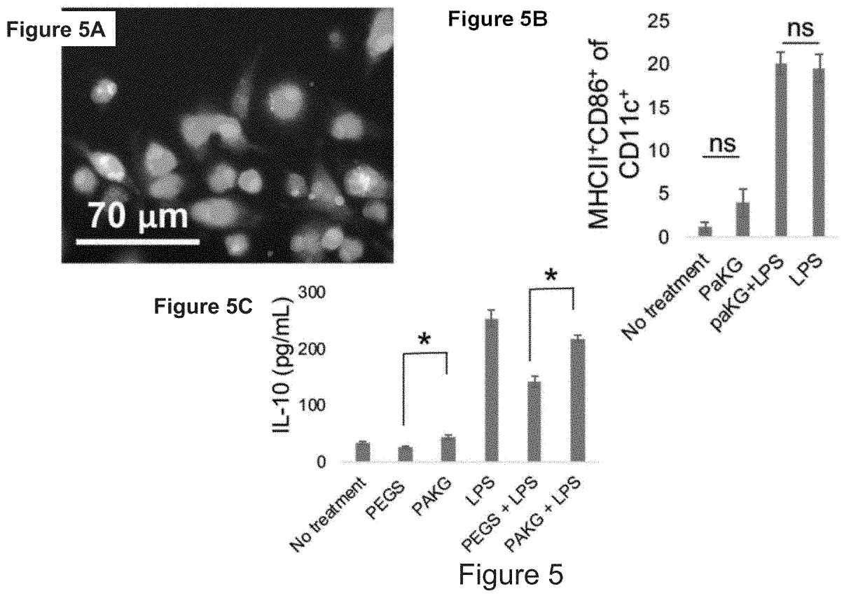

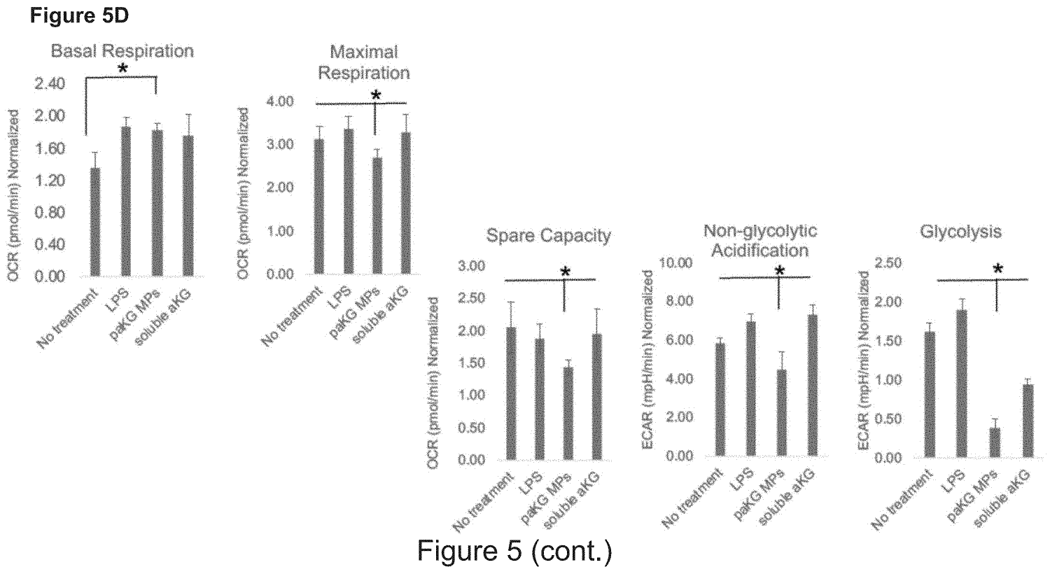

To generate immunosuppressive DCs, these cells should be able to express antigens and at the same time generate low levels of activating signals (co-stimulatory molecules/cytokines). To determine if intracellular sustained delivery of αKG can generate immunosuppressive DC phenotype (MHCII⁺CD86^{Lo}IL-10⁺IL-12p70^{Lo}), DCs were differentiated from bone marrow of C57BL/6j using a 10 day-protocol (>90% purity). After 2 hours incubation of rhodamine (representative drug) encapsulated pαKG particles with DCs, it was observed that DCs were able to phagocytose these particles (FIG. 4A and FIG. 5A).

Next, in order to determine if the particles, which are capable of releasing αKG induce activation in DCs, these cells were cultured with pαKG for 24 hours, and the cells were then stained for CD11c, CD86, and MHC-II. It was observed that the pαKG particles marginally increased activation in DCs as observed using flow cytometry (FIG. 4B and FIG. 5B). Moreover, IL-10 and IL-12p70 expression demonstrated that pαKG+lipopolysaccharide (LPS) group expressed nearly 2-fold higher levels of IL-10 as compared to LPS only control, but expressed 2.5-fold lower levels of IL-12p70 as compared to LPS only group (FIG. 4C, FIG. 4D, and FIG. 5C). Moreover, to test if pαKG can modulate adaptive immune responses, spleen derived CD3+ T-cells (magnetically separated) were added (10:1 ratio T-cells:DCs) to DCs cultured with the particles. These cells were cultured for 48 hours, stained for CD4, CD8, Tbet, CD25, Foxp3, GATA3, and analyzed by flow cytometry. It was observed that the pαKG particles alone substantially increased the ratios of Treg/Th1, Th2/Th1, and Treg/Tc1 as compared to the untreated control. These data strongly indicated that pαKG particles alone can be non-activating, and may even be immunosuppressive.

### Example 3: pαKG Polymers Upregulated Intracellular Metabolites Involved in Fatty Acid Oxidation (FAO) Pathway

In order to determine, which metabolic pathways are modulated upon culture with particles, DCs were seeded in 6-well plates and cultured with pαKG particles for 24 hours. No treatment and PLGA particles were utilized as control. The cells were then lysed and metabolites were analyzed using LC-MS. It was determined that the metabolites in the FAO pathways, such as sebacic acid, capric acid, glutaconic acid, heptadecanoic acid, hexanoic acid, decanoylcarnitine among others, were upregulated and glycolysis pathway metabolites were down-regulated in DCs cultured with pαKG as compared to the no treatment control, PLGA, and PEGS. These data strongly indicated that pαKG particles upregulate the intracellular levels of metabolites in the FAO pathway, which is the pathway utilized by immature or immunosuppressive DCs for energy.

### Example 4: pαKG Particles Accelerated Wound Healing in Mice by Reducing the Activation of Immune Cells in the Wound Bed

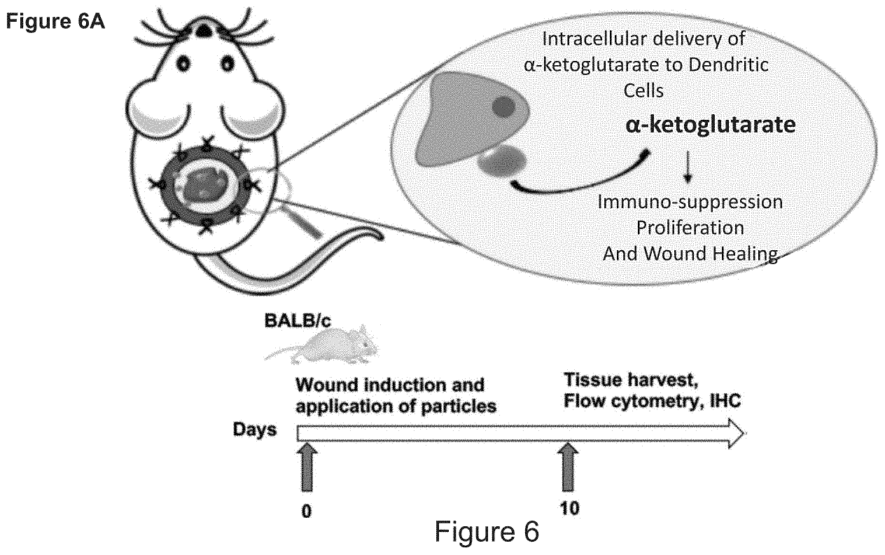

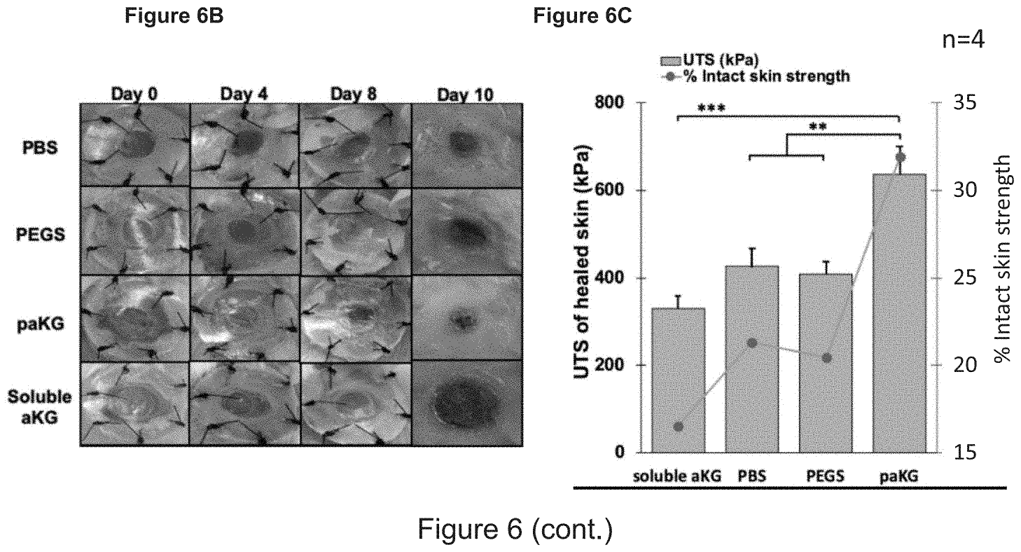

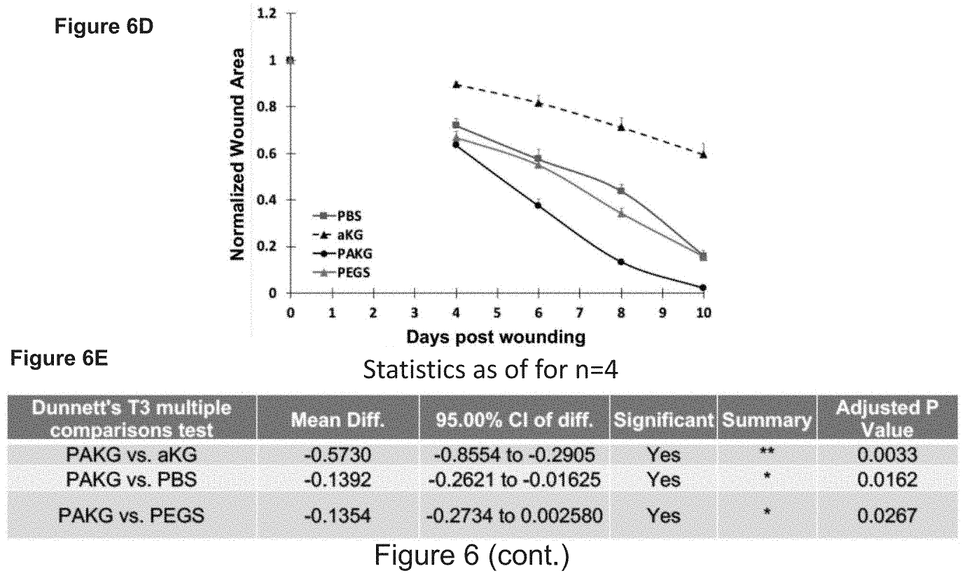

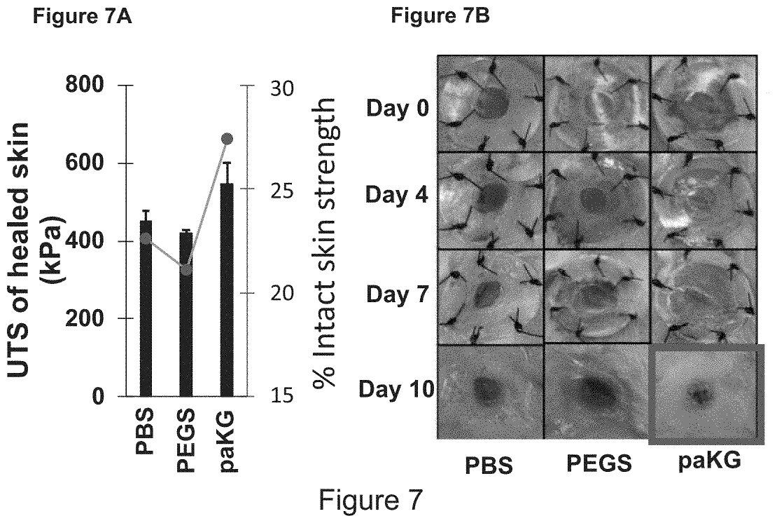

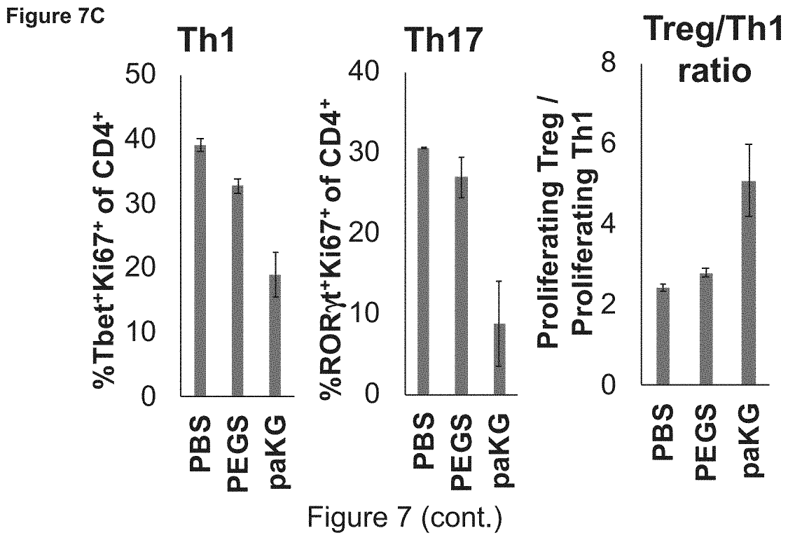

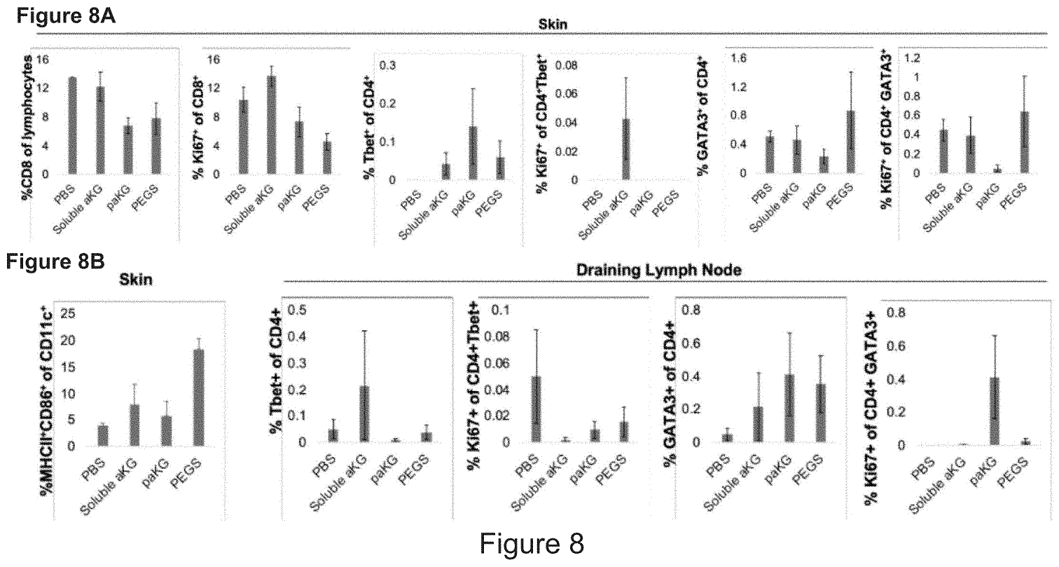

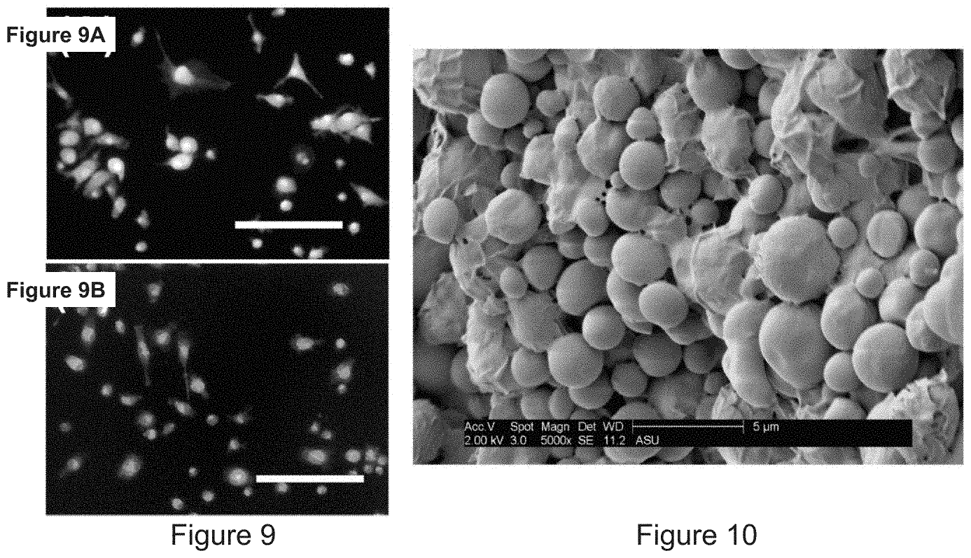

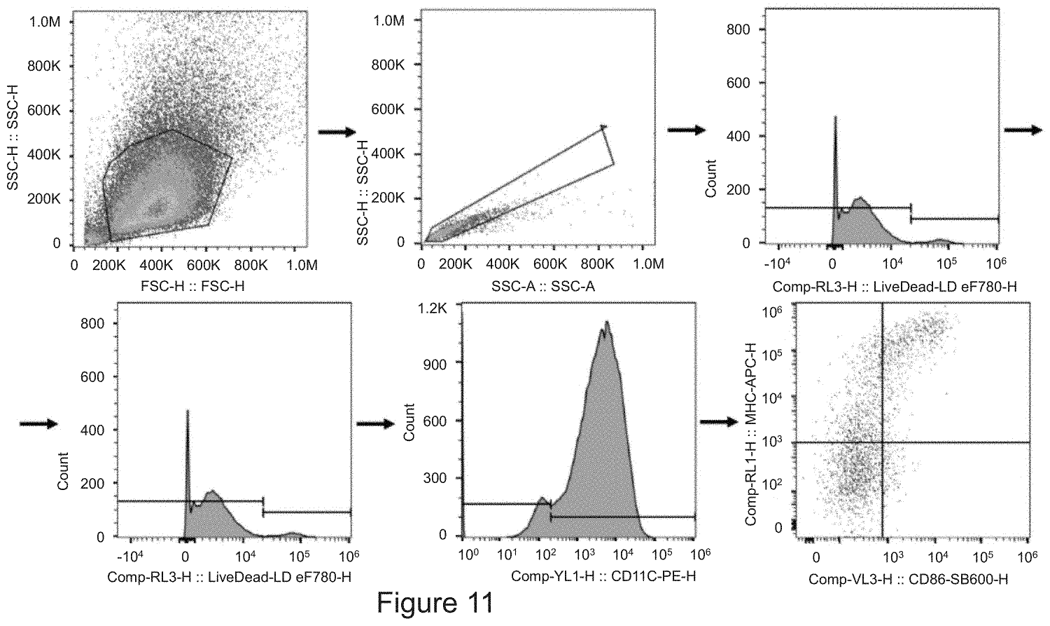

In order to reduce the risk of the described experiments, experiments were performed in a wound healing model and the effect of pαKG particles on wound healing rate was determined. Specifically, 2 BALB/c mice/group were utilized (pilot study), and a 5 mm wound was created on the back of the mice. Splints were applied to the wound to prevent them from closing. pαKG or PEGS particles or phosphate buffered saline (PBS) was added on top of the wound on day 0, which was then covered with Tegaderm dressing. Wound closure was observed for 10 days by taking photographs. On day 10, mice were sacrificed and skin was isolated (FIG. 6A). Ultimate tensile strength studies were performed on the skin to determine the strength of the healed skin. Moreover, frequency of Tregs, Th1, Th2, and Th17 in the skin tissue was determined using flow cytometry. It was observed that the ultimate tensile strength was higher in the pαKG group as compared to the PEGS (FIG. 6C and FIG. 7A), indicating effective wound closure. It was also observed that the pαKG particles led to faster wound closure as compared to no treatment control and PEGS (FIG. 6B and FIG. 7B—red box).

Additionally, the proliferating Th1 (CD4⁺Tbet⁺Ki67⁺) and Th17 (CD4⁺RORγt⁺Ki67⁺) populations in the skin were reduced and the ratio of proliferating Tregs to proliferating Th1 cells (CD4⁺CD25⁺Foxp3⁺Ki67⁺/CD4⁺Tbet⁺Ki67⁺) was significantly increased in pαKG group as compared to the PEGS and PBS group, which indicated that the inflammation was subsided that might have then lead to faster wound closure (FIG. 7C).

In summary, the above disclosed data demonstrated a method of making pαKG polymers and microparticles and use of the polymers and microparticles to accelerate wound healing. Mechanism of action is thought to be through induction of immune suppressive phenotype (i.e., decreased expression of pro-inflammatory cytokines, increased expression of anti-inflammatory cytokines, and increased T regulatory cells). The pa-KG polymers and microparticles were biodegradable, accelerated wound closure, and were useful for acute and chronic wounds.

1. A compound or salt thereof comprising the structure of Formula (I) 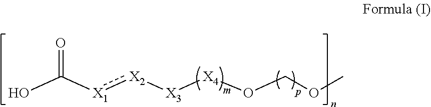
wherein each occurrence of X₁ and X₂ is independently C═R₁, CR₂, or CR₃R₄;
each occurrence of X₃ and X₄ is independently C═R₁ or CR₃R₄;
the bond between X₁ and X₂ is a single bond or a double bond;
wherein when the bond between X₁ and X₂ is a single bond, X₁ and X₂ are each independently C═R₁ or CR₃R₄, and when the bond between X₁ and X₂ is a double bond X₁ and X₂ are each CR₂;
R₁ is selected from the group consisting of O, NH, and S;
each occurrence of R₂, R₃, and R₄ is independently selected from the group consisting of hydrogen, hydroxyl, carboxyl, alkyl, cycloalkyl, heterocycloalkyl, aryl, and heteroaryl;
m is an integer represented by 0, 1, 2, or 5;
p is an integer from 1 to 50; and
n is an integer from 1 to 1000.

2. The compound of claim 1, wherein R₁ is O.

3-8. (canceled)

9. A particle comprising at least one compound of claim 1.

10. The particle of claim 9, wherein the particle has an average size of about 0.01 μm to about 1000 μm.

11. The particle of claim 9, wherein the particle encapsulates at least one therapeutic agent, wherein the compound comprising the structure of Formula (I) encapsulates the at least one therapeutic agent.

12. A method of delivering a therapeutic agent to a cell in a subject in need thereof, the method comprising administering at least one particle of claim 9 to the subject, wherein the particle encapsulates the therapeutic agent.

13. The method of claim 12, wherein the therapeutic agent is an anti-inflammatory therapeutic agent.

14. The method of claim 12, wherein the particle releases a therapeutic agent inside or outside the cell, wherein the therapeutic agent is a metabolite selected from the group consisting of α-ketoglutarate (αKG), succinic acid, citric acid, spermidine, itaconic acid, and any combination thereof.

15. The method of claim 12, wherein the particle is administered orally, topically, intravenously, intraperitoneally, or intramuscularly to the subject.

16. A method of enhancing biological tissue growth in a subject in need thereof, the method comprising administering at least one particle of claim 9 to the subject.

17. A method of enhancing biological wound healing or wound closure in a subject in need thereof, the method comprising administering at least one particle of claim 9 to the subject.

18. The method of claim 16, wherein the compound releases a therapeutic agent inside or outside the cell, wherein the therapeutic agent is a metabolite selected from the group consisting of αKG, succinic acid, citric acid, spermidine, itaconic acid, and any combination thereof.

19.-21. (canceled)

22. The particle of claim 11, wherein the therapeutic agent is a metabolite selected from the group consisting of αKG, succinic acid, citric acid, spermidine, itaconic acid, and any combination thereof.

23. The particle of claim 9, wherein the particle further comprises at least one therapeutic agent.

24. The particle of claim 23, wherein the therapeutic agent is a metabolite selected from the group consisting of αKG, succinic acid, citric acid, spermidine, itaconic acid, and any combination thereof.

25. The method of claim 12, wherein the therapeutic agent is a metabolite selected from the group consisting of αKG, succinic acid, citric acid, spermidine, itaconic acid, and any combination thereof.

26. The method of claim 16, wherein the particle further comprises at least one therapeutic agent.

27. The method of claim 26, wherein the therapeutic agent is a metabolite selected from the group consisting of αKG, succinic acid, citric acid, spermidine, itaconic acid, and any combination thereof.

28. The method of claim 17, wherein the particle further comprises at least one therapeutic agent.

29. The method of claim 28, wherein the therapeutic agent is a metabolite selected from the group consisting of αKG, succinic acid, citric acid, spermidine, itaconic acid, and any combination thereof.
